(19) United States
(12) Patent Application Publication
(10) Pub. No.: US 2022/0253797 A1
(43) Pub. Date: Aug. 11, 2022

(54) Methods, Systems, and Non-transitory Storage Media for Monitoring Progression of Medicinal Plants

(71) Applicants: Jessica Billingsley, Denver, CO (US); Amy Poinsett, Golden, CO (US)

(72) Inventors: Jessica Billingsley, Denver, CO (US); Amy Poinsett, Golden, CO (US)

(21) Appl. No.: 17/728,684

(22) Filed: Apr. 25, 2022

Related U.S. Application Data
(63) Continuation-in-part of application No. 13/092,188, filed on Apr. 22, 2011.

(51) Int. Cl. G06Q 10/08; G06F 21/60; G06F 21/62; G16H 10/60; G16H 20/90

(52) U.S. Cl. CPC G06Q 10/087; G06F 21/602; G16H 20/90; G16H 10/60; G06F 21/6245

(57) ABSTRACT

Information identifying a specific patient and disposal information associated with a specific medicinal plant is obtained, and the information identifying the specific patient is encrypted. Progression of the specific medicinal plant through multiple stages including planting, growth, application of an additive, harvesting, finishing, assembling into at least one product, packaging, and delivery is monitored in association with the specific patient. Disposal of at least a portion of the raw form of the specific medicinal plant and at least a portion of the product resulting from assembling the specific medicinal plant is tracked in association with the specific patient. Inventory of the specific medicinal plant is updated based on the disposal. The encrypted information identifying the specific patient is transmitted with the updated inventory information to a central server via a wireless network for maintenance in accordance with government regulations.

### CROSS REFERENCE

This application is a continuation-in-part of U.S. patent application Ser. No. 13/092,188, filed Apr. 22, 2011, herein incorporated by reference.

### TECHNICAL FIELD

The present disclosure relates generally to plant monitoring and, in particular, to monitoring progression of medicinal plants from seed to sale in association with specific consumers.

### BACKGROUND

A new industry is emerging for the sale of medicinal plants for medical purposes, such as cancer treatment and pain relief. Regulations regarding the sale of medicinal plants may vary from state to state and from community to community. Additionally, there are federal regulations for safeguarding patient records.

In other regulated product sales markets, such as the alcohol and drug markets, some amount of loss is anticipated and acceptable. In the medicinal plant market, such loss is not acceptable.

Currently, there is a need for an inventory control system for medicinal plants that sufficiently meets state/community/federal requirements in a manner that minimizes loss.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the present disclosure.

According to an exemplary embodiment, a method includes obtaining, by a scanner associated with a retail store, information identifying a specific patient and disposal information associated with a specific medicinal plant. The specific medicinal plant is identified by a unique plant id. The information identifying the specific patient is received from the scanner and encrypted, by a processor, in accordance with the Health Insurance Portability and Accountability Act (HIPAA). The specific medicinal plant is weighed at multiple stages of progression of the specific medicinal plant from a raw form, the multiple stages including planting, growth, application of an additive, harvesting, finishing, assembling into at least one product, packaging, and delivery. Progression of the specific medicinal plant through the multiple stages is monitored, by the processor, in association with the specific patient. Monitoring includes receiving, from the NTEP certified scale, a weight of the specific medicinal plant at each of the multiple stages. Disposal of at least a portion of the raw form of the specific medicinal plant and at least a portion of the product resulting from assembling the specific medicinal plant is tracked, by the processor, in association with the specific patient. Inventory of the specific medicinal plant is updated by the processor based on the disposal of the portion of the raw form of the specific medicinal plant and the portion of the product resulting from assembling the specific medicinal plant. The encrypted information identifying the specific patient and the updated inventory information are transmitted, by a transceiver, to a central server via a wireless network for maintenance in accordance with government regulations.

According to another embodiment, a system includes a scanner associated with a retail store. The scanner obtains information identifying a specific patient and disposal information associated with a specific medicinal plant, the specific medicinal plant identified by a unique plant id. The system further includes at least one National Type Evaluation Program (NTEP) certified scale to weigh the specific medicinal plant at multiple stages of progression of the specific medicinal plant from a raw form, the multiple stages including planting, growth, application of an additive, harvesting, finishing, assembling into at least one product, packaging, and delivery. The system further includes a processor and a memory having stored thereon instructions. The instructions, when executed by the processor, cause the processor to perform operations including receiving information from the scanner identifying the specific patient, encrypting the information identifying the specific patient in accordance with the Health Insurance Portability and Accountability Act (HIPAA), and monitoring, in association with the specific patient, progression of the specific medicinal plant through the multiple stages. Monitoring includes receiving, from the NTEP certified scale, a weight of the specific medicinal plant at each of the multiple stages. The operations further include tracking, in association with the specific patient, disposal of at least a portion of the raw form of the specific medicinal plant and at least a portion of the product resulting from assembling the specific medicinal plant. Tracking includes receiving, from the scanner, disposal information associated with at least a portion of the raw form of the specific medicinal plant and at least a portion of the product resulting from assembling the specific medicinal plant. The operations further include updating inventory of the specific medicinal plant based on the disposal of the portion of the raw form of the specific medicinal plant and the portion of the product resulting from assembling the specific medicinal plant. The system further includes a transceiver to transmit the encrypted information identifying the specific patient and updated inventory information to a central server via a wireless network for maintenance in accordance with government regulations.

According to another embodiment, a non-transitory storage medium has computer-executable instructions stored thereon which, when executed by a processor, cause the processor to perform operations. The operations include receiving, from a scanner associated with a retail store, information identifying a specific patient and disposal information associated with a specific medicinal plant. The operations further include encrypting the information identifying the specific patient in accordance with the Health Insurance Portability and Accountability Act (HIPAA) and monitoring, in association with the specific patient, progression of the specific medicinal plant, identified by a unique plant id, through multiple stages from a raw form. The multiple stages include planting, growth, application of an additive, harvesting, finishing, assembling into at least one product, packaging and delivery. Monitoring includes receiving, from at least one National Type Evaluation Program (NTEP) certified scale, a weight of the specific medicinal plant at each of the multiple stages. The operations further include tracking, in association with the specific patient, disposal of at least a portion of the raw form of the specific medicinal plant and at least a portion of the product resulting from assembling the specific medicinal plant, updating inventory of the specific medicinal plant based on the disposal of the portion of the raw form of the specific medicinal plant and the portion of the product resulting from assembling the specific product, and presenting the encrypted information identifying the specific patient and updated inventory information for transmission by a transceiver to a central server via a wireless network for maintenance in accordance with government regulations.

### BRIEF DESCRIPTION OF THE DRAWINGS

FIG. 1A illustrates an environment in which a system for monitoring progression of planta may be implemented according to an exemplary embodiment.

FIG. 1B illustrates a point of sale system according to an exemplary embodiment.

FIG. 2 illustrates an exemplary screen display for tracking transactions at a point of sale according to an exemplary embodiment.

FIGS. 3A-3E illustrate exemplary screen displays for tracking consumer records according to an exemplary embodiment.

FIGS. 4A-4E illustrate exemplary screen displays for product inventory tracking according to an exemplary embodiment.

FIGS. 4F-4R illustrate exemplary screen display for tracking administrative data concerning product inventory and sales transactions according to an exemplary embodiment.

FIGS. 4S and 4T illustrate exemplary screen displays for presenting product inventory and sales transaction reports according to an exemplary embodiment.

FIGS. 5A-5J illustrate exemplary screen displays for detailed tracking of progression of products from raw form through processing and distribution according to an exemplary embodiment.

FIG. 6 illustrates a device for monitoring progression of plants according to an exemplary embodiment.

FIG. 7 illustrates a method for monitoring progression of plants according to an exemplary embodiment.

### DETAILED DESCRIPTION

Detailed exemplary embodiments are disclosed herein. It must be understood that the embodiments described and illustrated are merely examples that may be embodied in various and alternative forms, and combinations thereof. As used herein, the word “exemplary” is used expansively to refer to embodiments that serve as examples or illustrations. The figures are not necessarily to scale and some features may be exaggerated or minimized to show details of particular components. Specific structural and functional details disclosed herein are not to be interpreted as limiting.

According to exemplary embodiments, an integrated solution is provided for inventory tracking, patient tracking, and point of sale management for products, such as medicinal plants. It should be appreciated, however, that the embodiments described herein are not limited to medicinal plants and patient tracking but may be applicable for tracking of inventory of products and consumer records in any environment. For example, the embodiments described herein may be useful for tracking inventory of plants that may have non-medicinal purposes and/or other products, such as tea, coffee, herbs, candy, etc. Medicinal plant inventory and patient record tracking are described in exemplary embodiments for illustrative purposes.

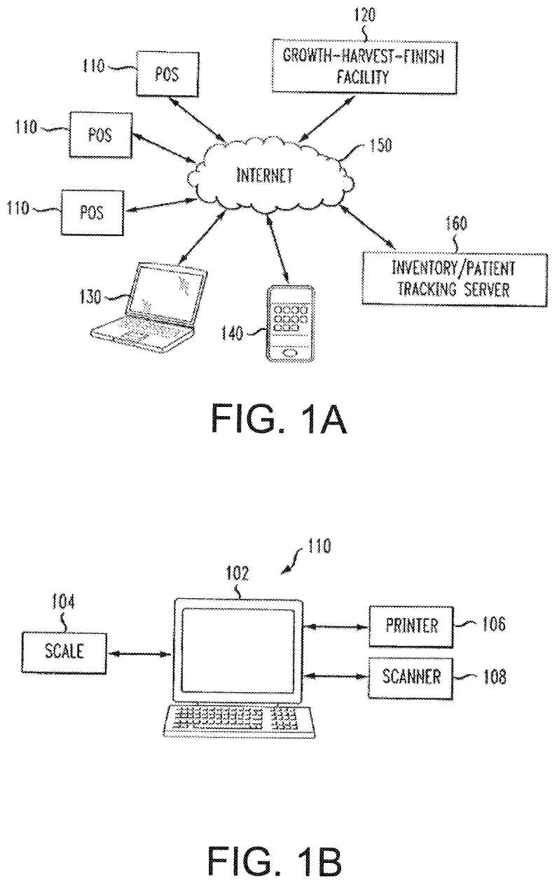

FIG. 1A illustrates an environment in which a system for tracking product inventory may be implemented according to an exemplary embodiment As shown in FIG. 1A, Point of Sale (POS) systems 110 are connected via a network 150, e.g., the Internet, to a remote inventory/patient tracking system 160. Though three POS systems 110 are shown for illustrative purposes, it should be appreciated that any number of POS systems may be implemented. Also connected to the inventory/patient tracking system 160 are a growth-harvest-finish facility or vendor 120 and end user end devices 130 and 140. Although only one growth-harvest-finish facility 120 and two end user devices 130 and 140 are shown for illustrative purposes, it should be appreciated that any number of growth-harvest-finish facilities 120 and end user devices 130 and 140 may be implemented. Also, although the growth-harvest-finish facility 120 is illustrated as being separate from the POS systems 110, all or part of the growth-harvest facility 120 may be collocated with the POS systems 110.

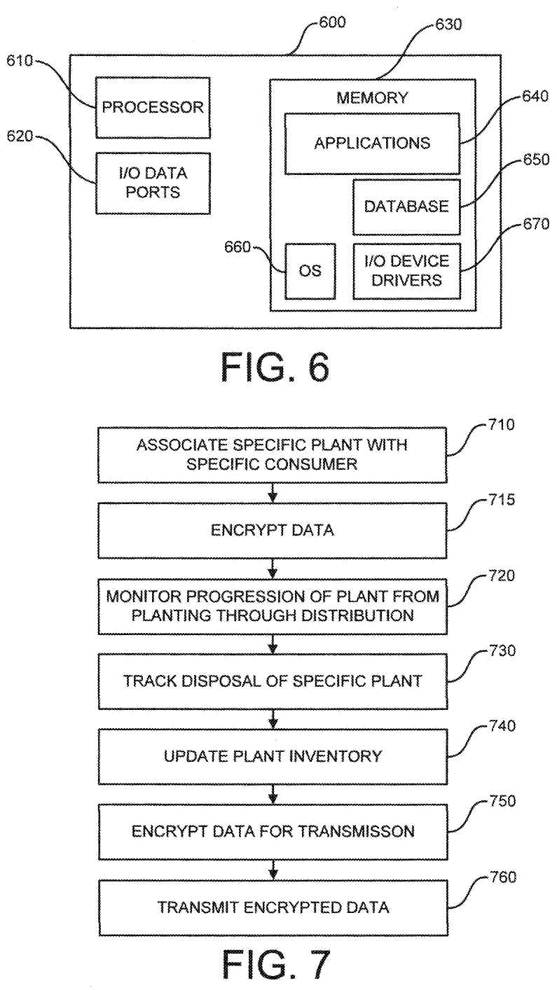

The network 150 may include a wireless Internet network, a wired Internet network, or a combination of both. The POS systems 110 may be implemented with a personal computer, printer, scanner and other components shown and described in further detail below with reference to FIG. 1B. The growth-harvest-finish facility 120 may be implemented with a personal computer, scanner, printer, scale, and other components, similar to the implementation of the POS systems 110 described below with reference to FIG. 1B. The end user devices 130 and 140 may include, e.g., personal computers, personal digital assistants, mobile communication devices, or any other type of communication device capable of communicating via the network 150 with the inventory/patient tracking system 160 (and the POS systems 110 and the growth-harvest-finish facility 120, as appropriate, e.g., for remote connection). The inventory/patient tracking system 160 may be implemented with a server or device, such as that illustrated and described with reference to FIG. 6 below. Each of the entities 110, 120, 130, 140, and 160 may communicate with each other via wired and/or wireless connections and the network 150.

According to exemplary embodiments, the POS systems 110 handle transactions for patients (herein defined as including caregivers and authorized representatives of patients) and account for inventory updates upon sale of products to patients. The growth-harvest-finish facility 120 handles progression of medicinal plants, from planting through distribution, in association with specific patients and accounts for the inventory of such medicinal plants through distribution. The end user devices 130 and 140 may be used by patients, caregivers, and/or other authorized users to inquire about inventory and/or patient records, initiate orders, update patient records, etc. The inventory/patient tracking server 160 maintains patient records and inventory records, accounting for patient record updates and inventory updates from the POS systems 110, the growth-harvest-finish facility 120, and the end user devices 130 and 140, in compliance with local, state, and federal regulations. The patient record updates and inventory updates may be exported to the inventory/patient tracking server 160 and/or retrieved by the inventory/patient tracking server, e.g., periodically or on demand. The growth-harvest-finish facility 120 and POS 110 also provide current information regarding patient records and inventory to each other and to the entities 130, 140, and 160. This is described in more detail below.

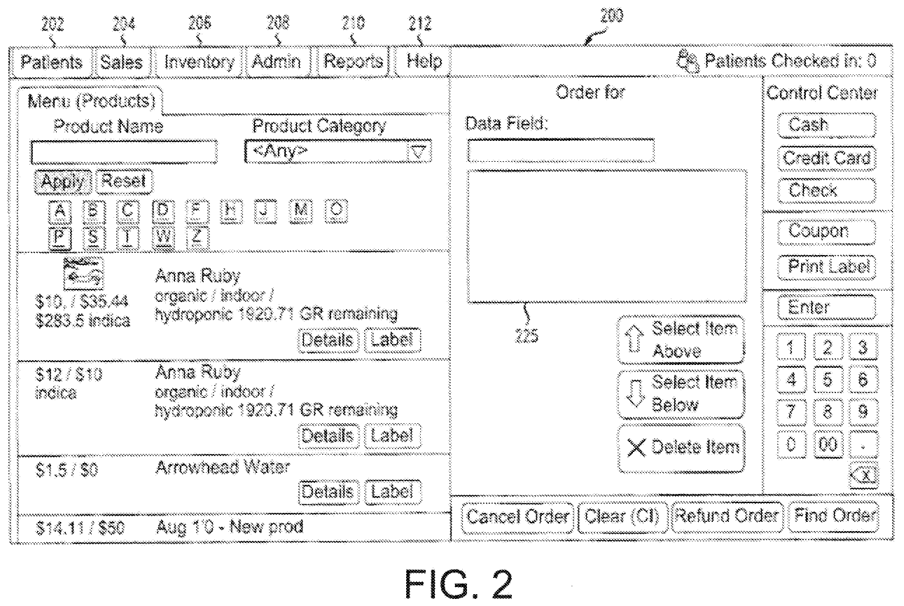

FIG. 1B illustrates an exemplary point of sale system in detail. The POS 110 includes a personal computer 102 or other device with which a user may access the network 150 to communicate with the growth-harvest-finish facility 120, the end user devices 130 and 140, and the inventory/patient tracking server 160. The personal computer may include components of a device 600 shown in FIG. 6 and described below. The personal computer 102 may also include a touchscreen for user interaction. A scale 104 for weighing a product made from processing a medicinal plant is connected to the personal computer 102. According to an exemplary embodiment, the scale 104 is implemented with a scale certified as a National Type Evaluation Program (NTEP) scale meeting the requirements established by the National Conference of Weights and Measures (NCWM). Also connected to the personal computer 102 are a printer 106 for printing, e.g., customer receipts and product labels, and a scanner 108, such as a magnetic strip and/or bar code scanner. The scanner 108 is used for scanning information, such as patient identification from, e.g., a magnetic strip on a driver's license. The scanner 108 may also be used for scanning product label information, e.g., two dimensional DL bar codes, RFID tags, etc. Although illustrated as one component, it should be appreciated that multiple scanners may be implemented for scanning, e.g., magnetic strips, bar codes, and RFID tags. The manner in which the personal computer 102 is used in conjunction with the scale 104, the printer 106, and the scanner 108 is described in more detail below with reference to FIG. 2.

Although not illustrated, the growth-harvest-finish facility 120 may include similar components as the POS 110, e.g., a scale for weighing plants, portions of plants, and/or products resulting from processing of plants, a scanner for scanning bar codes associated with plants, a personal computer for tracking plant inventory, etc.

FIGS. 2-5J illustrate various screen displays which may be accessed by a user with a username and password. The user's access may be timed such that the user is automatically logged out after a predetermined amount of time. The user's access may also be tracked and time-stamped, and different users may be given different levels of access. For example, an administrator may be given full access, while an employee, patient, or other user may be given limited access.

FIG. 2 illustrates a screen display 200 which may be used at a POS 110 according to an exemplary embodiment. The screen display 200 may be accessed by a user, e.g., an employee, at the POS 110. Using the screen display 200, a user may select a product for sale to a patient, charge the patient, track the amount of product sold, track the price charged for the product, and deduct the amount of product sold from inventory, all in compliance with local, state, and federal regulations.

Referring to FIG. 2, the user at the POS 110 is presented with a screen including, e.g., a patients tab 202, a sales tab 204, an inventory tab 206, an admin tab 208, a reports tab 210, and a help tab 212. Upon selecting the sales tab 204, the user is presented with the screen 200. Depending on the patient's needs and prescription, the user may use a menu to select a product by name and/or category. For example, the user may select a particular strain, category and type of cultivation for a plant, select a weight of product for sale, and select a price.

According to exemplary embodiments, the weight of product selected may be automatically populated by weighing the product on the integrated scale 104 at the POS 110. The price may be prepopulated or customized, e.g., by the user. The product may be priced strictly according to weight and/or different weights may be priced taking into account popularity of various weights. For example, a popular weight of product may be sold at a price that is different than the strict price for volume of the product. The price may be adjusted to account for specials and discounts, which may vary by patient and by the amount of product being sold. For example, a patient that is a frequent buyer may be given a 10% member's discount, and a patient that buys a large quantity may be given a bulk discount. In addition, the price may be adjusted such that it is slightly less than or more than the price indicated for the amount of product weighed. For example, if the amount of product weighs slightly more than a standard weight, the price may be adjusted down to the price for the closest standard weight, so that the patient is only charged for the closest standard weight down from the actual weight. The price paid is accounted for separately from the weight, such that the actual price paid for and the actual weight of the product are both accurately tracked.

Additional products and hard goods may be selected for sale to the patient. Such products may include other medicinal plant products and non-regulated products. Information identifying products to add to a patient's order may be manually entered or scanned in via the scanner 108, e.g., by scanning a bar code attached to product packaging. Products may be prepackaged, e.g., by weight, or products may be custom-made, e.g., weighed out to a custom weight.

As products are added to a patient's order, a summary of the types and amounts of products sold, the price for the products (including sales tax), and information identifying the products may be provided in section 225.

The amount due from a patient for an order may be automatically populated by swiping, e.g., a credit card or debit card, via the scanner 108. Or, if payment is tendered via a check or cash, the amount may be manually entered using a touchscreen keypad. If a coupon is applicable, this may be indicated via the screen 200. The summary information appearing in section 225 and product labels may be printed. The summary information may be provided in a receipt to the patient, and product labels, e.g., for medicinal bottles or other containers for the medicinal plant products, may be provided by printing on the printer 106 upon checkout.

Although not illustrated in detail, the screen 200 may also display an order history which may be used by the user to select a patient and review information identifying the patient. The order history shows what products the patient has ordered in the past, what amount of products have been ordered, etc. The order history may be used, e.g., to re-print a receipt, find a past order for a patient such that the user may duplicate all or portions of the order without reentering all the information for the order, etc.

The screen 200 may also include a calculator by which a determination may be made as to how much of a certain product may be bought with a certain amount of money. Thus, a user at the POS 110 may enter an amount of money and see how many grams or ounces of a particular product can be purchased for that amount. This information may also be accessible by patient/caregiver using a device 130 and/or 140 via, e.g., remote communication with the POS 110 and/or via a website obtaining up to date information, e.g., from the inventory/patient tracking system 160.

According to an exemplary embodiment, upon completion of a sale, the amount(s) of medicinal product sold (which is accounted for as a disposal) is deducted from the inventory, and the price paid for the product and the patient to which the product sold are accounted for. Non-medicinal product sales may also be accounted for. Such information is provided to the inventory/patient tracking system 160, where it is maintained in compliance with local and state regulations, e.g., Colorado's HB 1284.

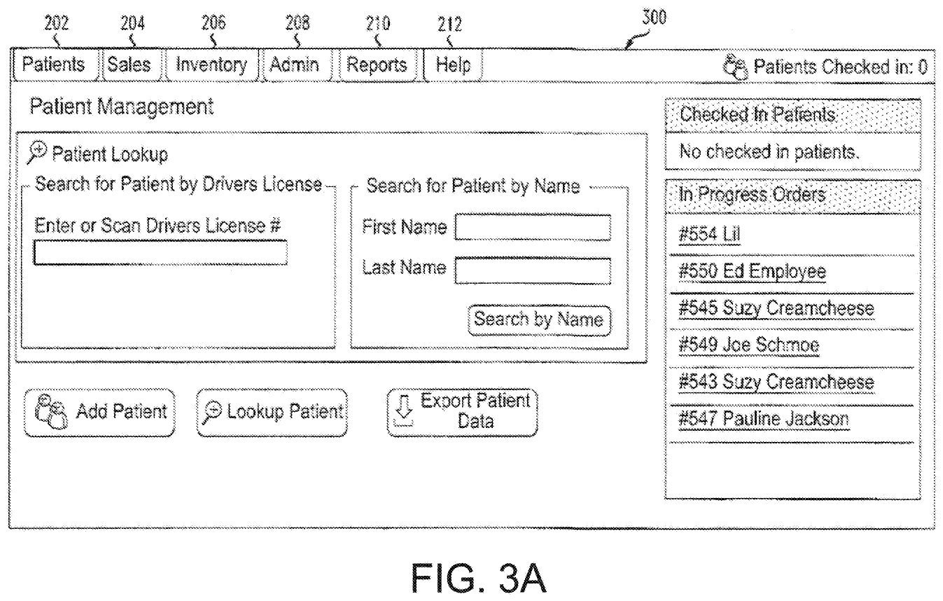

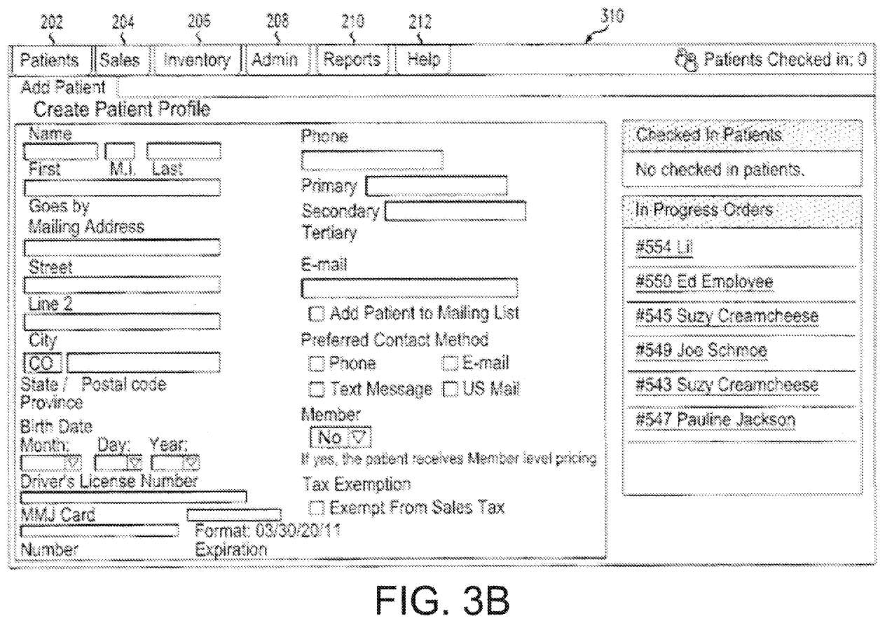

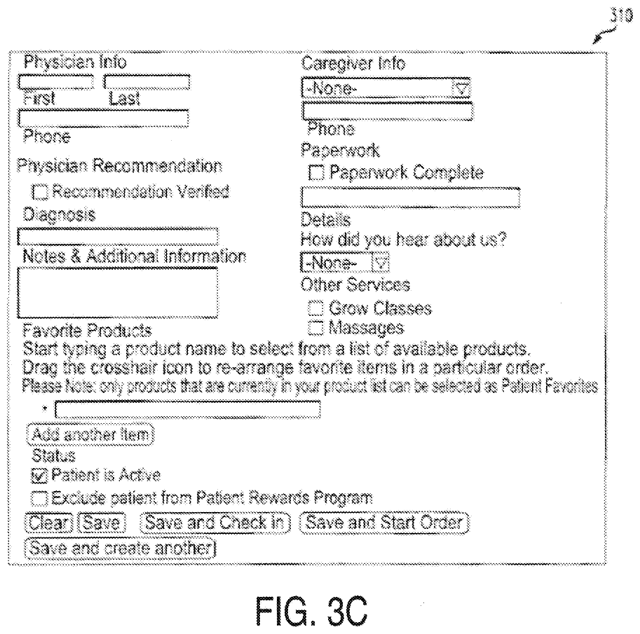

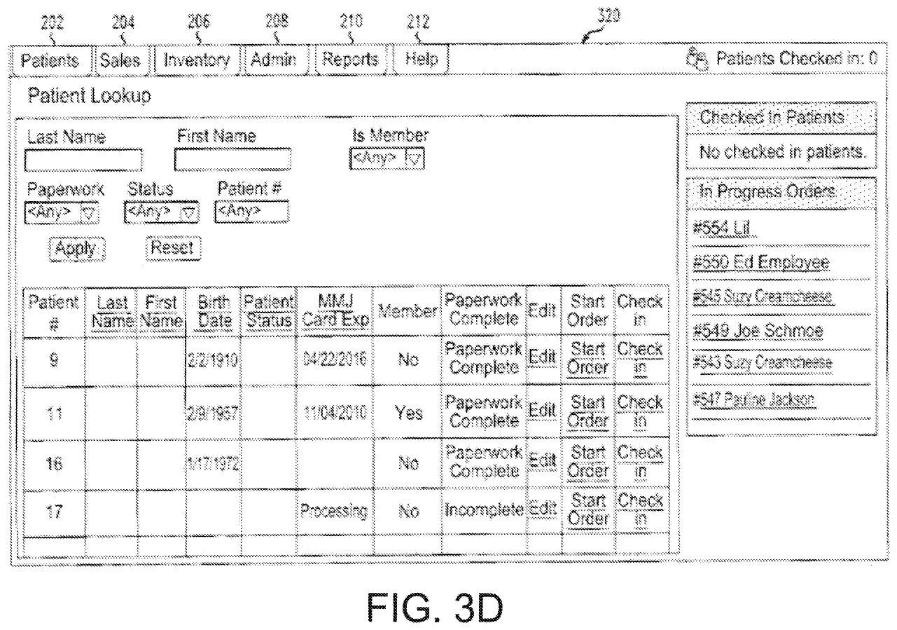

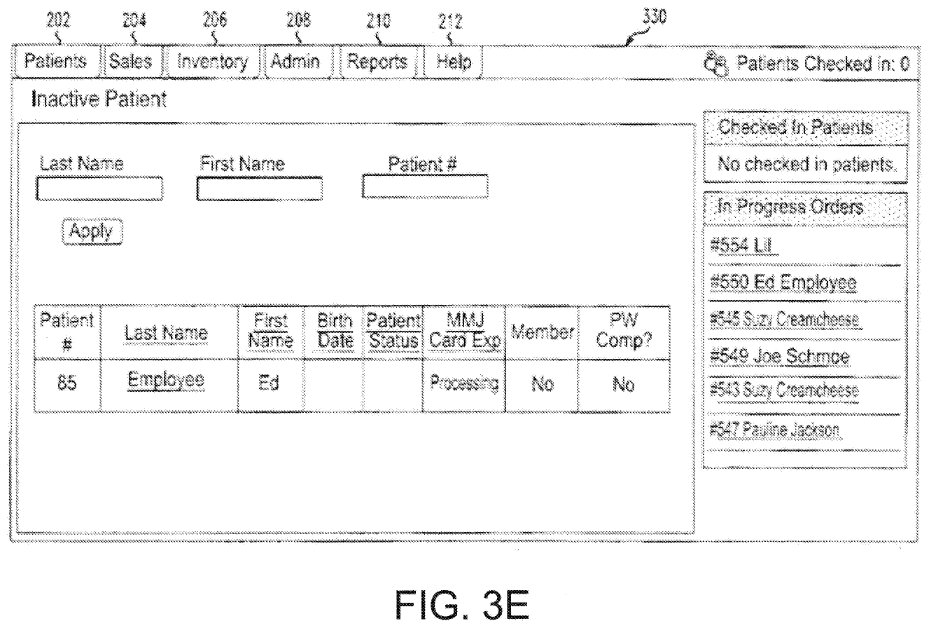

FIGS. 3A-3E illustrate exemplary screen displays that may be used for tracking patient data according to an exemplary embodiment. The screen displays shown in FIGS. 3A-3E may be accessed by a user at a POS 110 and/or by a user, e.g., a patient, caregiver, or authorized user, via devices 130, 140, etc.

Referring to FIG. 3A, by selecting a patients tab 202, a user may be presented with tabs (not shown) for adding a patient, looking up an active patient, and managing inactive patients. According to an exemplary embodiment, selection of the tab 202 may cause the user to be presented with a screen display 300 which may be used for patient management. The screen display 300 provides options for adding a patient and looking up an active patient. Checked in patients and in progress orders are also indicated on the screen display 300. Using screen display 300, a patient may be checked in such that a sales transaction as described above may be initiated.

Upon selection of an add patient option via the screen display 300 or selection of an “add patient” tab by the user, the screen display 310 shown in FIGS. 3B and 3C is presented. Using this screen, the user may populate information for a new patient, e.g., by manually entering the information or by scanning. e.g., an identification card, at the POS 110 or any other suitable location. Information that may be populated for the patient may include, e.g., the patient's name, mailing address and telephone number(s), the prescribing physician and/or caregiver's names and telephone number(s), the patient's birthday, information regarding the patient's diagnosis for which a product is prescribed, insurance information, favorite products, preferred method of contact, an indication of whether the patient is an active patient and/or a member of a rewards program, other services for the patient, etc. The patient's personal information may be populated by a user at the POS 110 and/or by the patient or another authorized user, based on access given to the user, patient, and/or caregiver. Notes may be added, e.g., by a user at the POS 110, for the patient. The patient's personal information may be entered manually or automatically, e.g., by scanning an identification card, e.g., driver's license. Scanning may be performed by the scanner 108, which may scan, e.g., magnetic strip, two dimensional DL barcodes, etc.

Once a patient is added, the patient may be looked up by selecting the “patient lookup” tab or the patient lookup option via the screen 300 causing the screen display 320 shown in FIG. 31) to appear. Using the screen display 320, the patient may be looked up by name, patient number, etc. The patient may also be looked up by scanning, e.g., a driver's license, at the POS 110. The user may select a patient from a list of patients, start an order for a patient, and check the patient in using the screen 320. As shown in FIG. 3D, information displayed for a looked patient may include, e.g., the name, birthdate, patient status, identification card expiration, membership status, an indication of paperwork status, etc. Additional information that may be displayed for a looked up patient may include, personal patient information such as that shown in FIGS. 3B and 3C, notes regarding a patient, patient history indicating dates of visits, an expiration date of patient's identification card, an image of the patient, etc. The patient information may be modifiable via the screen 320.

According to an exemplary embodiment, the screen 320 may be used to manage active patients. Inactive patients may also be managed via selection of an inactive patients tab (not shown). Referring to FIG. 3E, an inactive patient may be looked up, e.g., by name and/or patient number. The inactive/active status of the patient may be updated via the screen 330 and/or the screen 320.

As shown in FIGS. 3A-3E, the status of patients, e.g., checked in or not checked-in, may be indicated in each of the screens 300-330. Once a patient is checked in, a plant tender at a growth-harvest-finish facility 120 may be notified.

According to exemplary embodiments, the patient data populated on the screens 300-330 is safeguarded according to state regulations. e.g., Colorado's HB 1284, and federal regulations, such as the Health Insurance Portability and Accountability Act (HIPAA). Such safeguards may include encrypting the patient data using a key and encryption ciphers to convert the patient data into ciphertext. Examples of ciphers that may be used include: Key Exchange Algorithms (RSA, DH, ECDH, DHE, ECDHE, PSK), Authentication/Digital Signature Algorithm (RSA, ECDSA, DSA), Bulk Encryption Algorithms (AES, CHACHA20, Camellia, ARIA), and Message Authentication Code Algorithms (SHA-256, POLY1305).

These ciphers may be used in combination as cypher suites. Examples of cipher suites include:

TLS_AES_256_GCM_SHA384

TLS_CHACHA20_POLY1305_SHA256

TLS_AES_128_GCM_SHA256

TLS_AES_128_CCM_8_SHA256

TLS_AES_128_CCM_SHA25

The patient data may be further encrypted for transmission. According to an exemplary embodiment, an encryption protocol, such as Transport Layer Security (TLS) or Secure Sockets Layer (SSL), may be used to safeguard transmission of the patient data using an asymmetric public key infrastructure. The patient data may be encrypted with a public key before transmission, to, e.g., the inventory/patient tracking system 160. Transmitted patient data may be decrypted by, e.g., the inventory/patient tracking system 160 using a private key.

Similar encryption may be applied for storage and transmission of plant inventory data, item inventory data, sales data, etc.

Data for each patient, with safeguards such as encryption applied, is transmitted to the inventory/patient tracking system 160 via an “export patient data” option on screen 300 shown in FIG. 3A. Alternatively or in addition, the inventory/patient tracking system 160 may retrieve the patient data, with safeguards such as encryption applied. The patient information may be maintained at the inventory/patient tracking system 160 in accordance with government regulations.

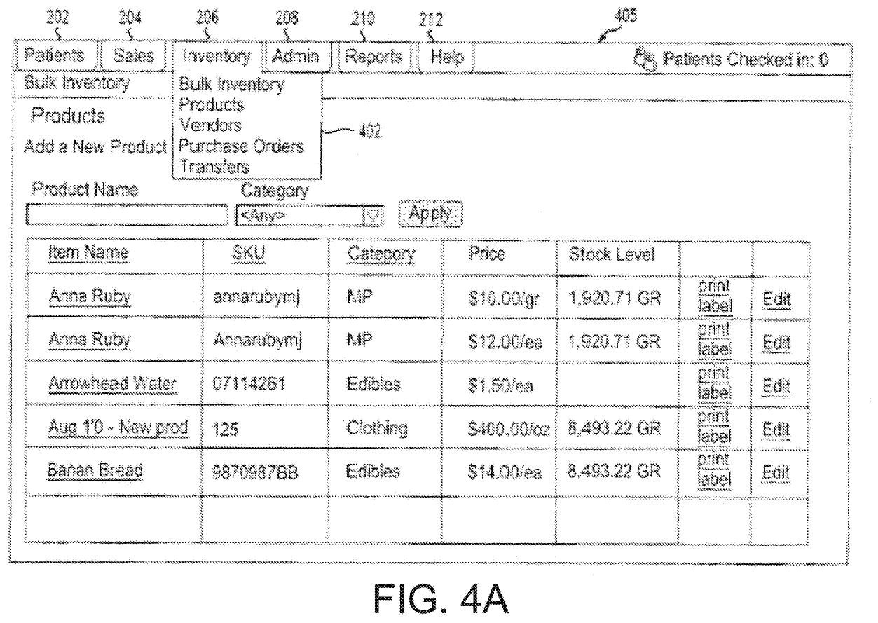

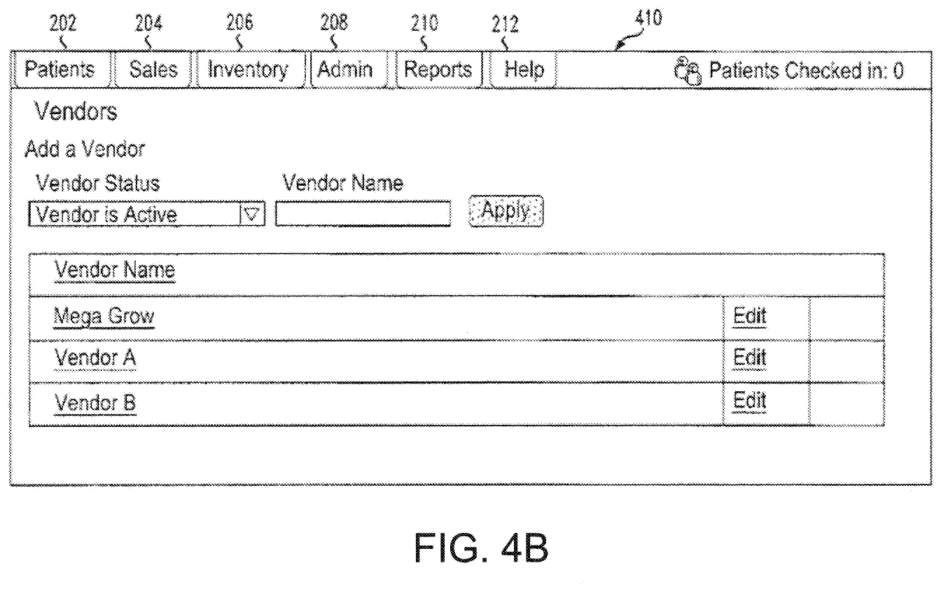

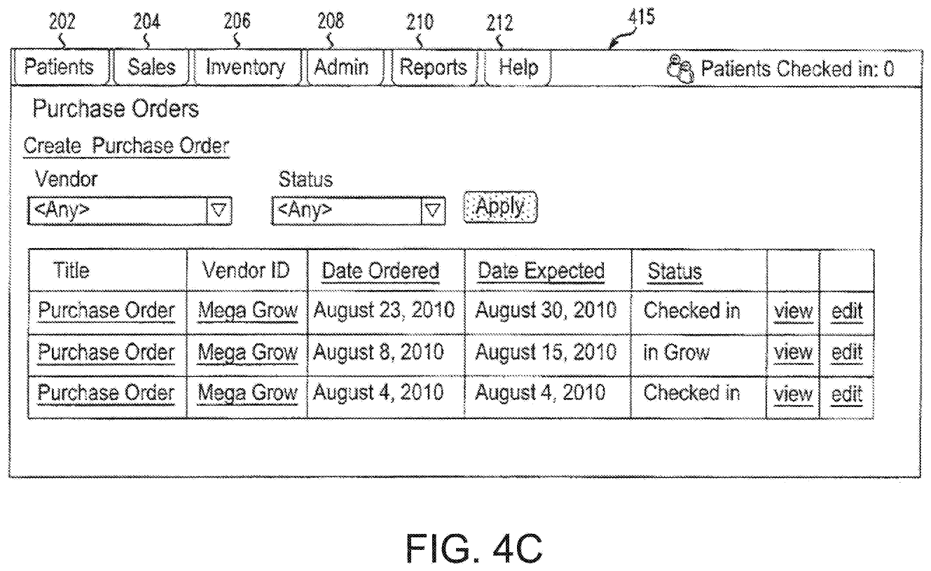

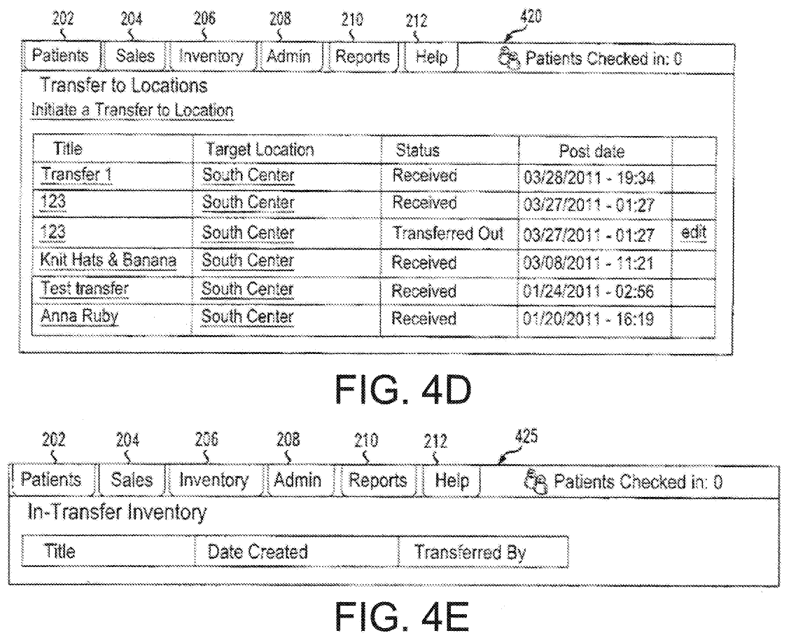

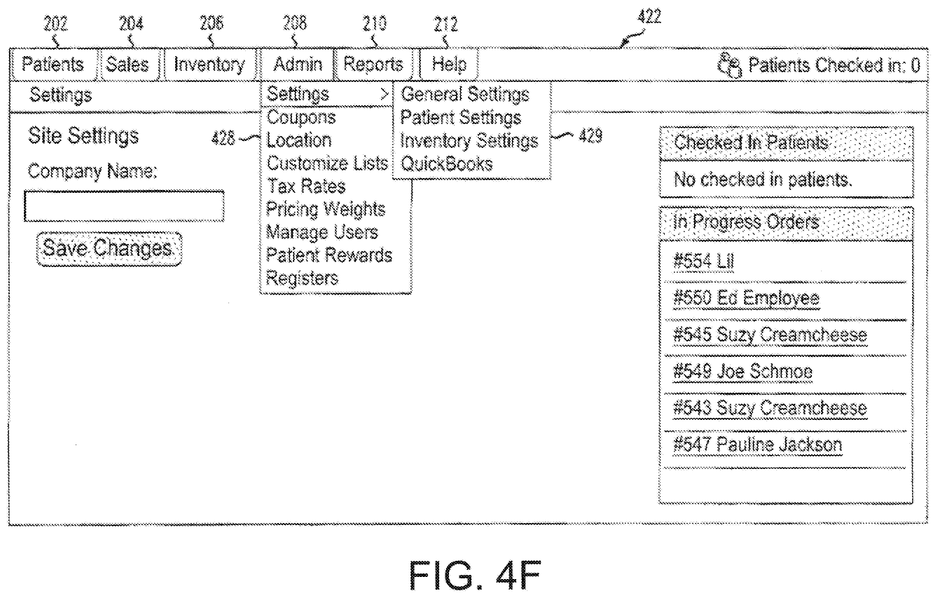

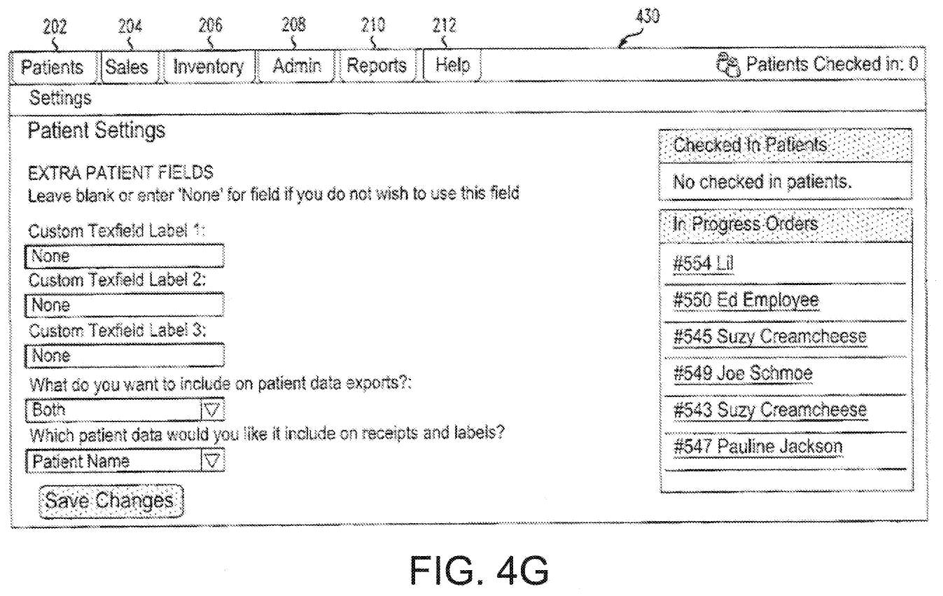

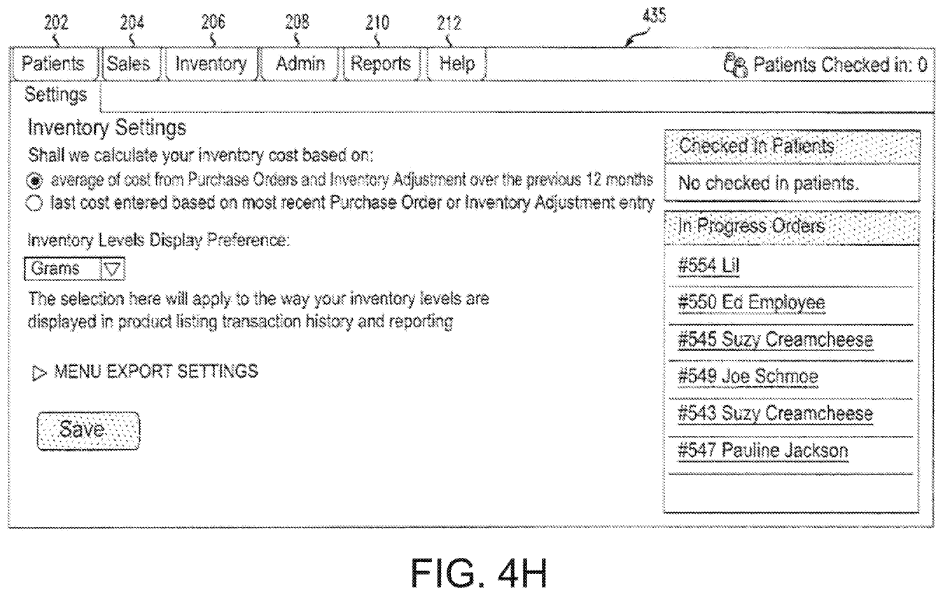

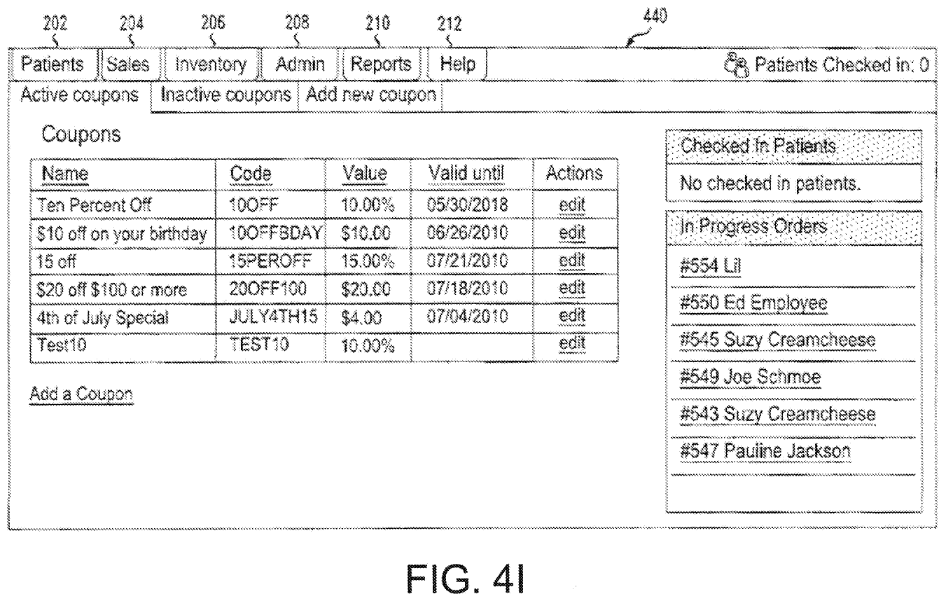

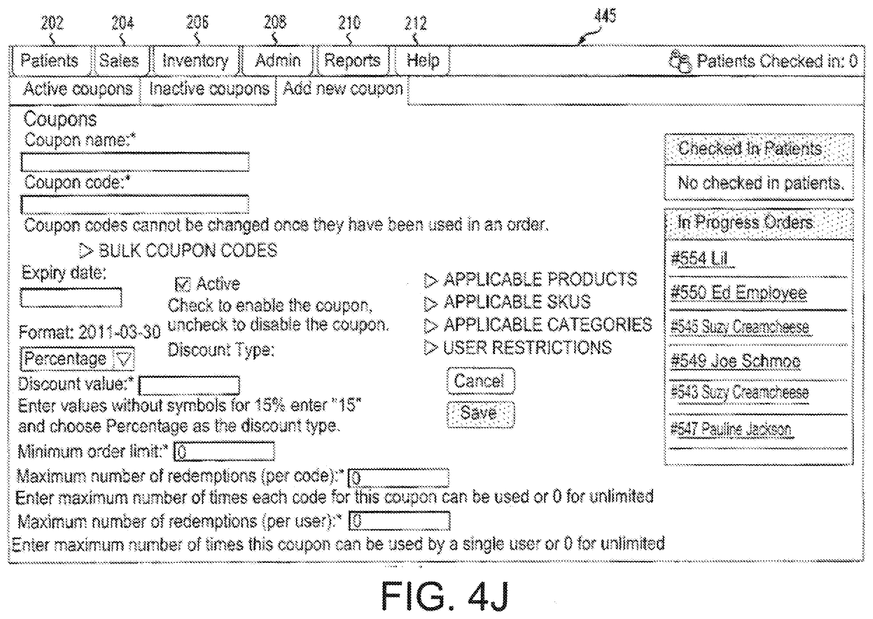

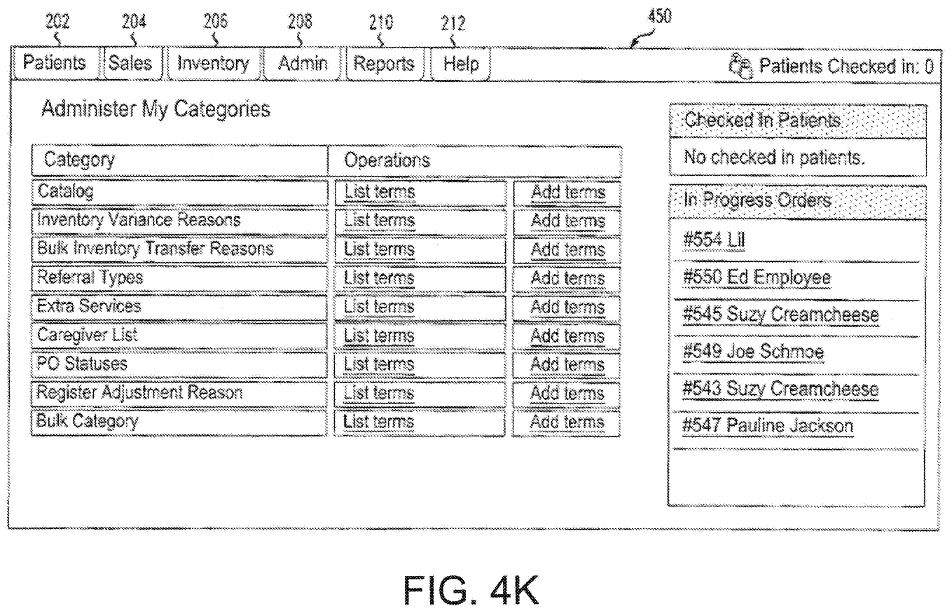

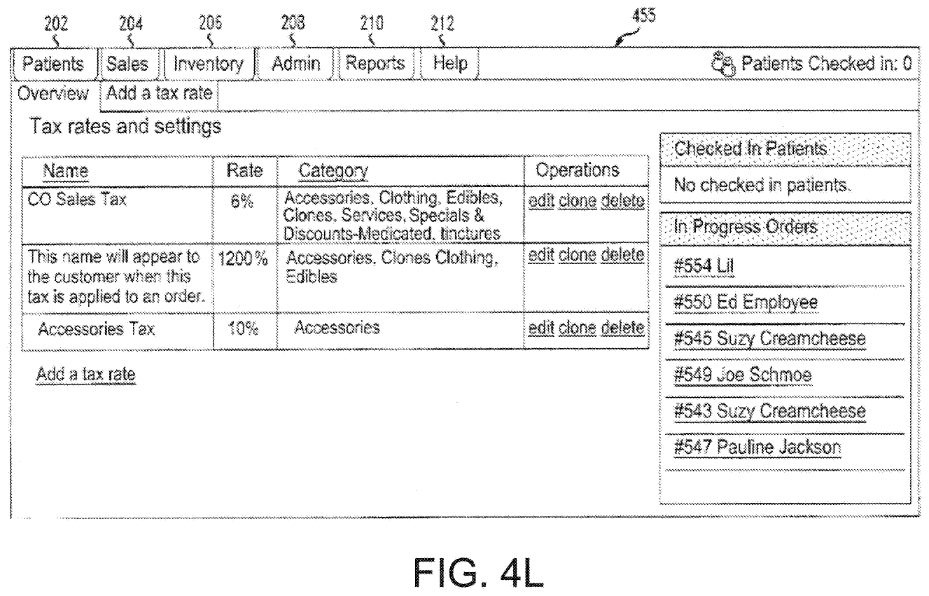

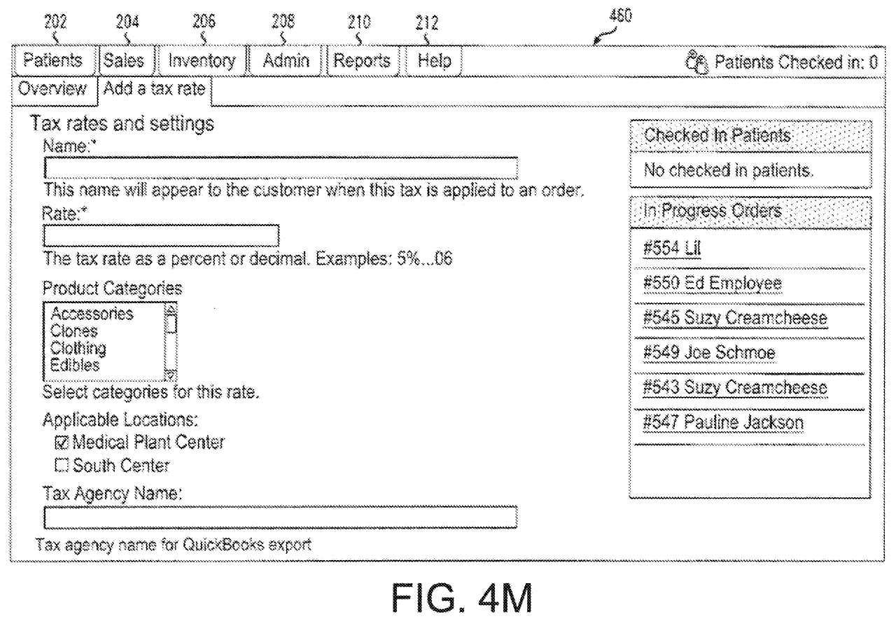

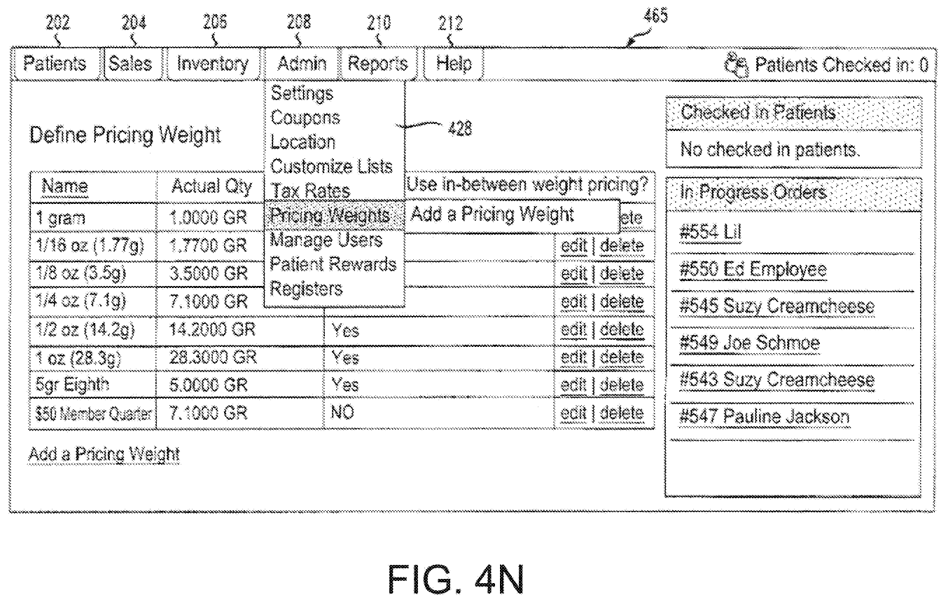

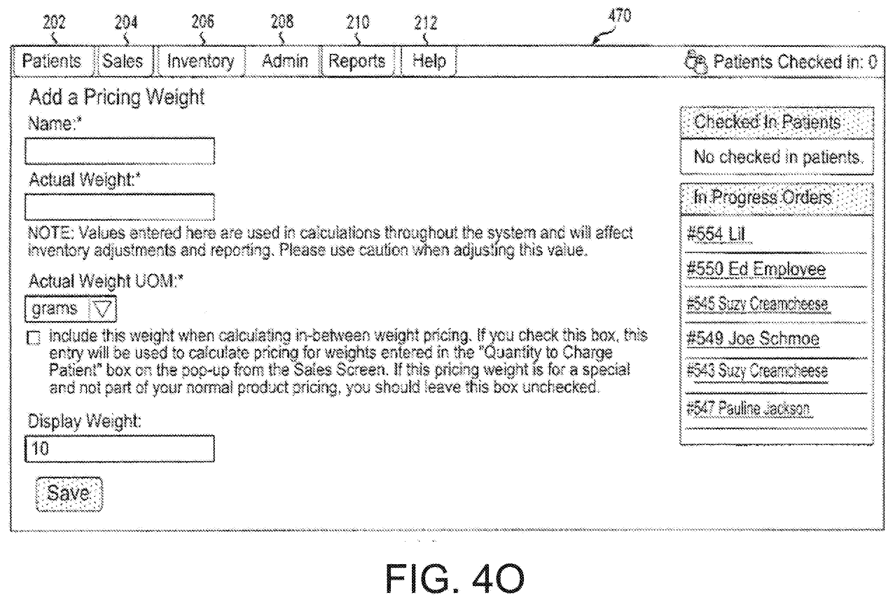

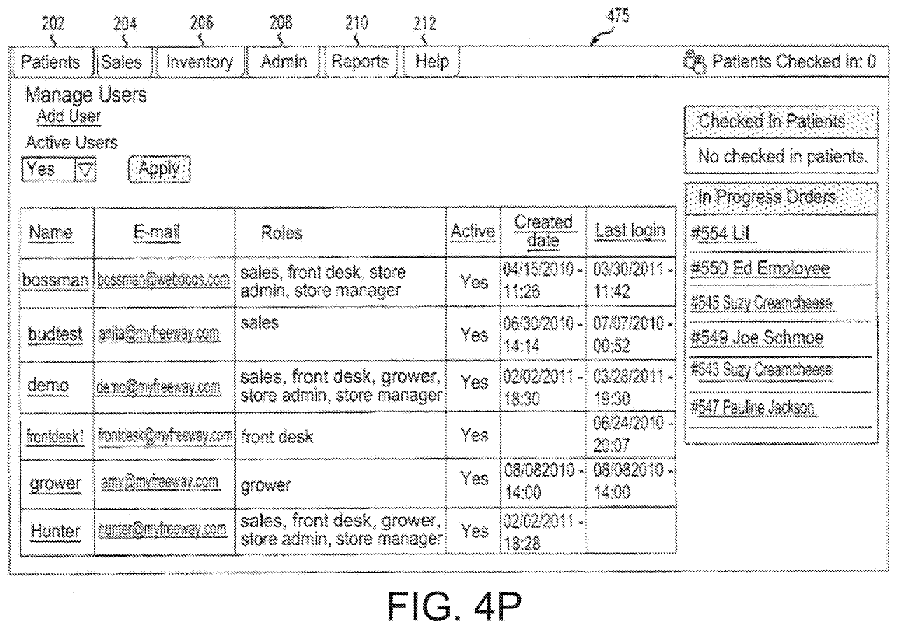

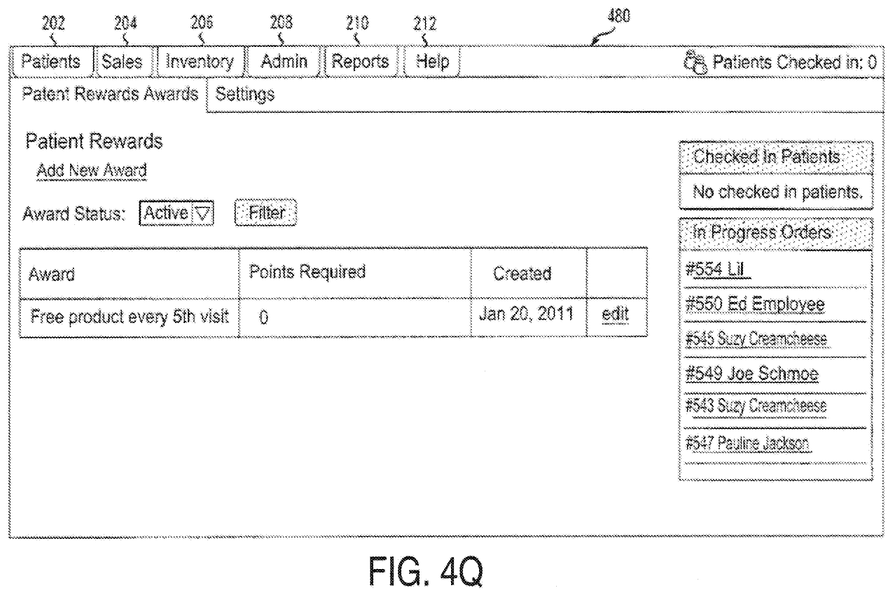

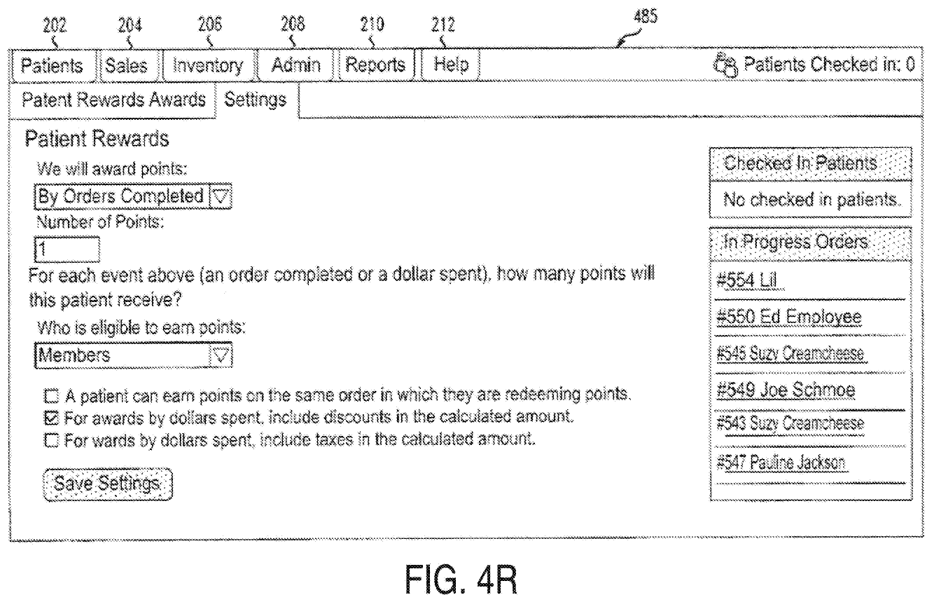

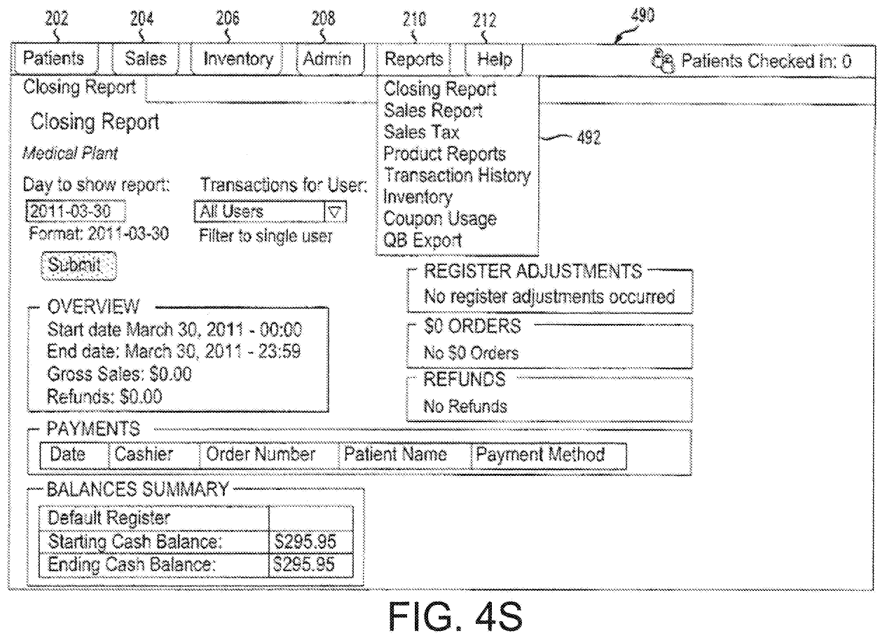

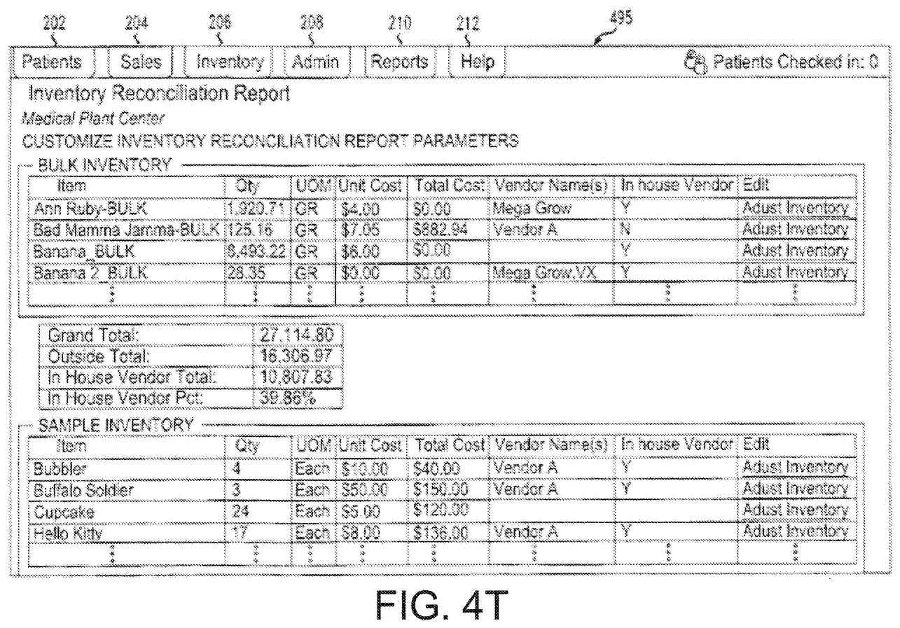

FIG. 4A-4E illustrate exemplary screen displays that may be used for tracking plant inventory according to an exemplary embodiment. By selecting the inventory tab 206, a user, e.g., at a POS 110, is presented with options for selecting inventory management options, e.g., bulk inventory, products, vendors, purchase orders and transfers via a tab 402. Selecting “bulk inventory” or “products” causes a screen, such as the screen display 405 shown in FIG. 4A to be presented to the user. Using this screen, the user may add a new product, search for a product, and review details regarding products, name, id (e.g., SKU or UPC), category, price (e.g., per gram or ounce) and stock level of the products (e.g., in grams or ounces). The details regarding products may also include, e.g., the date the product is received, the expiration date, and data indicating the quality/condition of the product. An image of the item may be uploaded and viewed via this screen, and notes regarding the item may be entered. The product information is modifiable and may be printed, e.g., via the printer 106, via user interaction with the screen display 405.

Selecting “vendors” via the tab 402 causes the screen display 410 in FIG. 4B to be displayed. Using this screen, a user may search for, modify, review, and add vendor information, including e.g., vendor status and name. The vendor information reflects the status of, e.g., the growth-harvest-finish facility 120.

Selecting “purchase orders” via the tab 402 causes the screen display 415 in FIG. 4C to be displayed. Using this screen, a user may search for, modify, review, and add purchase order information. The purchase order information may include, e.g., a vendor id, an order date, a date expected, and a status.

Selecting “transfers” via the tab 402 causes the screen display 420 shown in FIG. 4D to be displayed. Using this screen, a user may initiate a transfer of inventory from one location to another and may review and modify information regarding inventory transfers. This information may include, e.g., a target location, a status, and a post date.

Although not shown, the tab 402 may also include an option for receiving transfers. By selecting this option, the screen 425 shown in FIG. 4E may be displayed to user. Using this screen, a user may review information regarding inventory that is in the process of being transferred, including, e.g., the date the transfer was created and the entity initiating the transfer.

The inventory information reflected on the screen displays 400-425 is tracked and maintained by the inventory/patient tracking system 160. Upon entry of a new item and/or modification of information regarding an item, the inventory information at the POS 110 is updated, and the updated inventory information is transmitted to and/or retrieved by the inventory/patient tracking system 160.

Referring to FIG. 4F, selection of the admin tab 208 causes tabs 428 and 429 to be displayed for a user, e.g., a user at the POS 110, for reviewing and modifying administrative information. Tab 428 includes an option for settings, coupons, location, list customization, tax rates, pricing weights, user management, patient rewards, and registers. By selecting the “settings” option, the tab 429 is displayed with options for general settings, patient settings, inventory settings, and an accounting spreadsheet, e.g., QuickBooks or any other suitable accounting program spreadsheet. Selection of the “general settings” option also causes the screen display 422 shown in FIG. 4F to be presented to the user, e.g., a user at the POS 110. This screen allows the user to enter and update site settings, e.g., a company name, for a site to which the administrative information pertains.

Selection of the “patient settings” option via the tab 429 causes the screen display 430 shown in FIG. 4G to be presented to the user. Using this screen, the user may customize patient settings. e.g., information to be included on patient reports and patient information to be printed on receipts and labels.

Selection of the “inventory settings” option via the tab 429 causes the screen 435 shown in FIG. 411 to be presented. Using this screen, the user may indicate what to base inventory cost on. For example, the user may indicate that the inventory cost should be based on the average of costs from purchase orders and inventory adjustment over the previous year or that the cost be based on the last cost entered based on the most recent purchase order or inventory adjustment entry. The user may also select a preference for displaying inventory levels, e.g., in grams or ounces.

Although not shown, selection of the “QuickBooks” option via the tab 429 may case a screen display to be presented which may be used to import data into accounts set up in QuickBooks. The data imported may include, e.g., the names and types of sales, discounts, and COGS accounts. The data may also include COGS account adjustments, which may be used to track variations between the actual product weight and the quantity of product sold to a patient. The data may also include the names and types of bulk inventory, product inventory, and inventory adjustment accounts, the names and types of undeposited funds and register adjustment accounts, and the names and types of receivable accounts, payable accounts, and shipping/expenses accounts. This data may be exported at the account level or by subaccounts as desired by the user.

Referring again to FIG. 4F, selection of the “coupons” option causes tabs to be presented to user for active coupons, inactive coupons and add a new coupon. Selection of the “active coupons” tab causes the screen display 440 shown in FIG. 41 to be presented to the user. Using this screen, the user may review and modify details regarding active coupons, e.g., for a POS 110. Such details may include, e.g., the names of coupons, codes to enter for coupons, values of the coupons, and expiration dates. Although not shown, selecting the “inactive coupons” tab may cause a screen to be presented to the user with similar modifiable details regarding inactive coupons.

Selecting the “add a coupon” tab causes the screen display 445 shown in FIG. 4J to be presented to the user. Using this screen, the user may add information regarding a new coupon, including, e.g., the coupon name, code, expiration date, and discount type. A minimum order limit and/or maximum number of redemptions (per user or per code) may also be entered. Information regarding applicable products, applicable product ids, applicable categories of products, and user restrictions may also be entered. Once added, information regarding the new coupon may be reviewed via the screen 440.

Referring again to FIG. 4F, selection of the “location” option via the tab 428 may cause a screen display to be presented to the user with information regarding locations, e.g., a POS location 110. Although not shown, this screen display may include, e.g., a location name, contact, name and contact information, e.g., an email address. Such information may be modifiable by the user.

Selection of the “customize lists” option via the tab 428 causes the screen display 450 shown in FIG. 4K to be presented Using this screen the user may administer categories of information, e.g., catalog, inventory variance reasons, etc., and operations for the categories, e.g., terms for the categories. Terms may be added by the user via the screen 450.

Selection of the “tax rates” option via the tab 428 causes tabs for a tax overview and adding a tax rate to be presented. Selection of the “overview” tab causes the screen display 455 shown in FIG. 4L to be presented. Using this screen, the user may review and modify tax rate information including, e.g., names of taxes, rates, and categories of products to which the taxes apply.

Selection of the “add a tax rate” tab causes the screen display 460 shown in FIG. 4M to be presented. Information entered when adding a tax rate may include, e.g., the name of the tax, the rate, the categories of products to which the tax applies, locations to which the tax applies, and the tax agency name.

Selection of the “pricing weights” option via tab 428 causes the screen 465 shown in FIG. 4N to be presented. Using this screen, the user may review and modify weights by which products are priced by name and by actual quantity of the product. The user may indicate whether to use “in-between” weight pricing, e.g., when the product weight upon sale to a patient is in-between the defined pricing weights. For in-between weights, the user may indicate that the price be adjusted to price for the nearest defined weight that is lower than the actual weight.

Selection of an “add a pricing weight” option causes the screen 470 shown in FIG. 4O to be presented. Using this screen, the user may add a pricing weight for a product by entering the name of the product, the actual weight of the product to which the price applies, the actual weight unit of measurement (UOM) e.g., in grams, ounces, or pounds, the actual weight for in-between weight pricing (if applicable), and the weight to be displayed, e.g., at the POS 110.

Referring again to FIG. 4F, selection of the “manage users” option via the tab 428 causes the screen display 475 shown in FIG. 4P to be presented. Using this screen, a user, e.g., a supervisor or administrator may manage users that have access to various screens. User information that may be reviewed may include, e.g., the user name, contact information (e.g., email address), user role, status (active/inactive), date of creation of the user, and the user's last login date/time. This screen allows an authorized user to keep track of access by other users.

Selection of the “patients rewards” option via the tab 428 causes tabs for patient rewards awarded and settings to be presented. Selection of the “patient rewards awards” tab causes the screen display 480 shown in FIG. 4Q to be presented. Using this screen, the user may review and modify patient reward information including, e.g., the award description, qualifications for the reward (e.g., points required for the award), and the date the awarded was created.

Selection of the settings tab causes the screen display 485 shown in FIG. 4R to be presented. Using this screen, the user may enter settings for the awards, e.g., events for which points are awarded, the number of points that may be awarded for each event, and individuals/entities that are eligible to earn points. The user may also include whether a patient can earn points on the same order in which points are redeemed, whether discounts are included for awards by dollars spent, and whether taxes are included in the awards for dollars spent.

Although not illustrated, selection of the “registers option” via the tab 428 may cause a screen to be presented with information for registers, e.g., at a POS 110. The information may include, e.g., the names of registers and the corresponding register balances. The balances may be adjusted, and information regarding balance adjustment history of registers may be accessed.

The administrative information reflected on the screen displays 430-485 is tracked and maintained by the inventory/patient tracking system 160. Upon entry and/or modification of information via the screen displays 430-485, the administrative information at, e.g., the POS 110 is updated, and the updated information is transmitted to and/or retrieved by the inventory/patient tracking system 160.

Referring to FIG. 4S, selection of the report tab 210 causes the tab 492 to be presented for reviewing and modifying reports concerning transactions, inventory, and patient information. The tab 492 includes options for a closing report, sales report, sales tax, product reports, transaction history, inventory, coupon usage, and accounting program, e.g., QuickBooks (QB), export.

Selection of the “closing report” option causes the screen 490 shown in FIG. 4S to be displayed. Using this screen, the user may review a closing report for a POS 110, including information such as the date, transactions for particular users, register adjustments, $0 orders, refunds, gross sales, payments, and balances summary (starting cash balance and ending cash balance) for one or more registers

Although not shown, selection of the “sales report” option via the tab 492 may cause one or more screens to be presented with sales report information including, e.g., a sales report summary, sales report information by year, a custom sales report summary, order details, patients visits, and transactions. The sale report summary may include, e.g., the total number of orders, total revenue, and average order. This summary information may be presented, e.g., as sales by day and/or by year to date, daily averages, and projected yearly totals. Statistics for grand total sales, total active patients, and patients with whom transactions occurred on a given day may also be presented. In addition, the total orders may be broken down by status, e.g., POS transaction completed, payment received, pending, POS transactions in progress, in checkout, and POS canceled. Sales report information by year may include, e.g., the number of orders, the total revenue, and the average order by month for a given year. The custom sales report summary may include additional information, e.g. the number of orders, the number of products sold, and the total revenue for a particular date range.

Selection of the “sales tax” option via the tab 492 may cause a screen to be displayed including sales tax information (not shown). This information may include, e.g., a start date, an end date, order status, and jurisdiction.

Selection of the “product reports” option via the tab 492 may cause a screen to be displayed including product information (not shown). This information may include, e.g., product sales information, product listings, and products by weight/variation. The product sales information may include, e.g., name, category, quantity “on hand” (e.g., at a POS 110), actual weight, quantity sold, cost, discounts, gross revenue, and net income. This information may be presented for bulk inventory, as well. The product listing information may include, e.g., product name, category, description, price, id (e.g., SKU or UPC), status, and system id. The products by weight/variation information may include, e.g., product variation (for products with different variations), category, line item price, and number sold for each product.

Selection of the “transaction history” option via the tab 492 may cause a screen to be displayed including information regarding transaction history (not shown). This information may be customized by, e.g., title, type, date, transaction amount, adjustment, adjusted level, cost, and transactor.

Selection of the “inventory” option via the tab 492 causes the screen display 495 shown in FIG. 4T to be presented. Using this screen, the user may review information for reconciling inventory, including bulk inventory information reported by item name, quantity, UOM, unit cost, total cost, vender name, and an indication as to whether the vendor is in-house. The user may adjust the inventory as appropriate. Inventory may be reported as, e.g., bulk inventory and sample inventory.

Information presented upon selection of the options via the tab 492 may be exported, e.g., to a CSV file. This information is also maintained by the inventory/patient tracking system 160.

According to an exemplary embodiment, using the screens 2-5T, real time information may be accessed, e.g., by a user at a POS 110 and/or a user at an end user device 130 and/or 140 in remote connection with the POS 110. Some of this information, e.g., inventory information, may also be published in real time to a website to indicate inventory in stock, which may be accessible, e.g., by a patient/caregiver via an end user device 130 and/or 140. Such a website may obtain up to date information, e.g., from the inventory/patient tracking system 160 via the network 150.

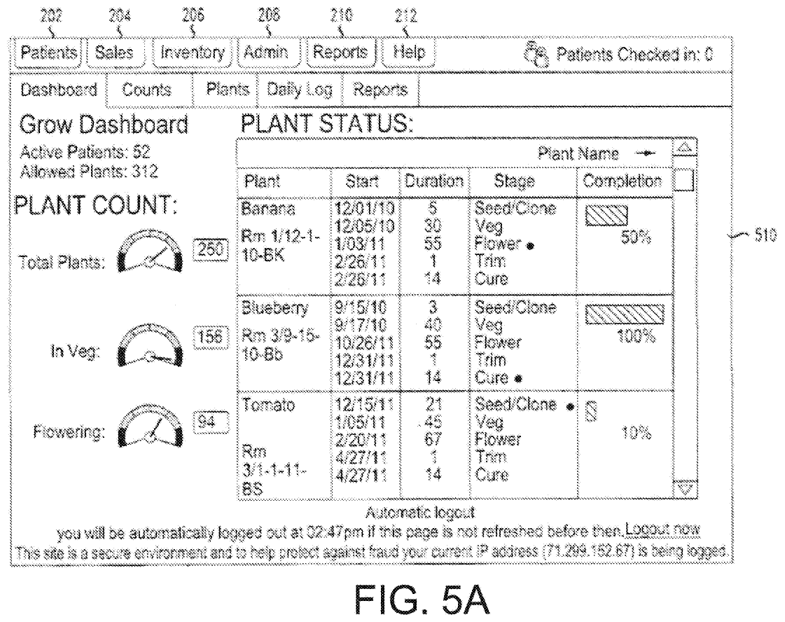

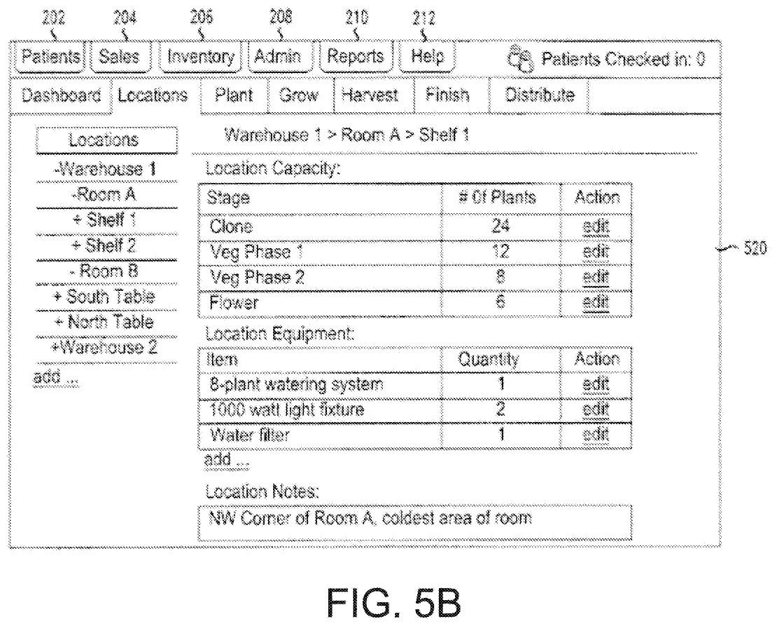

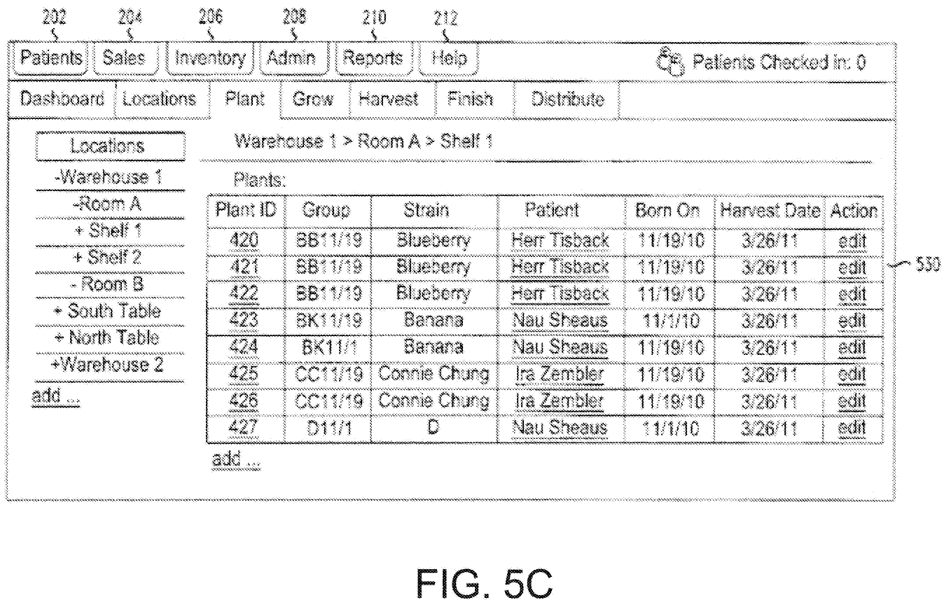

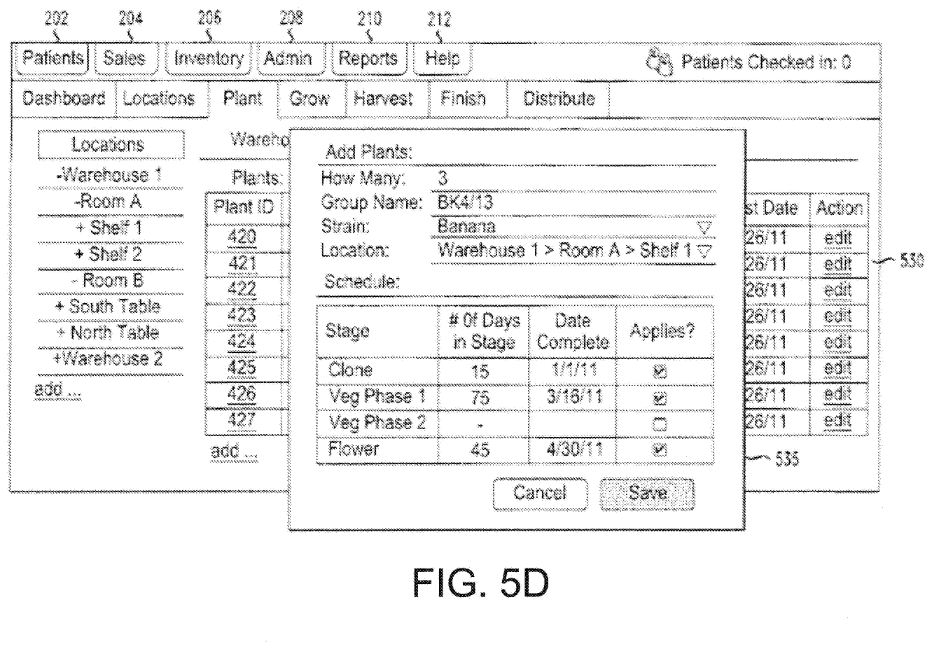

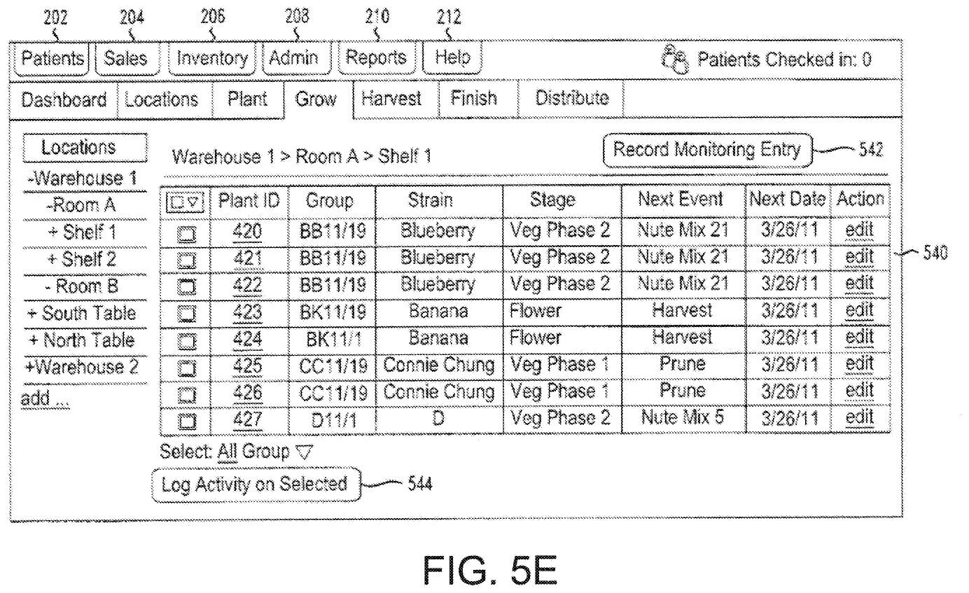

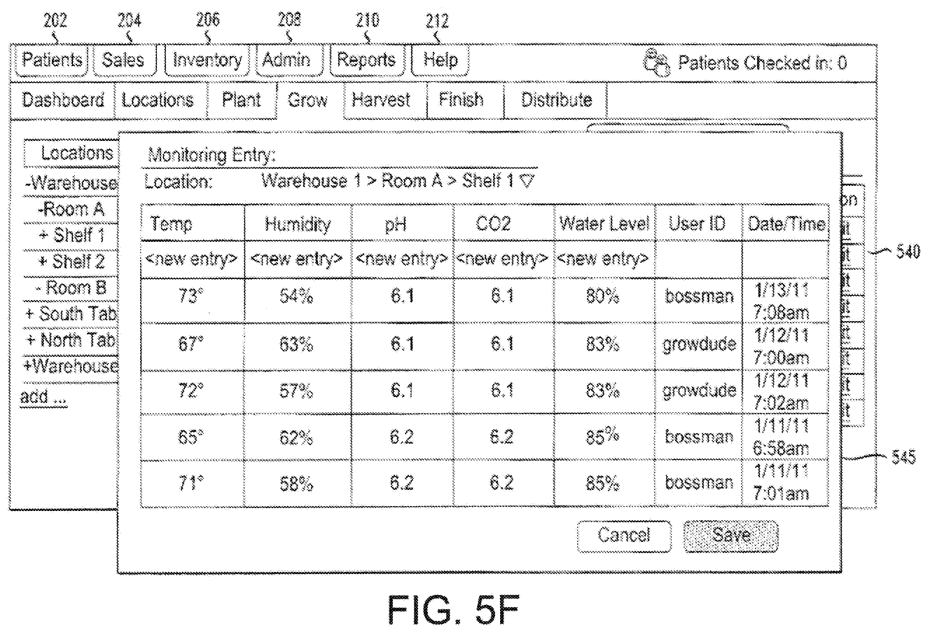

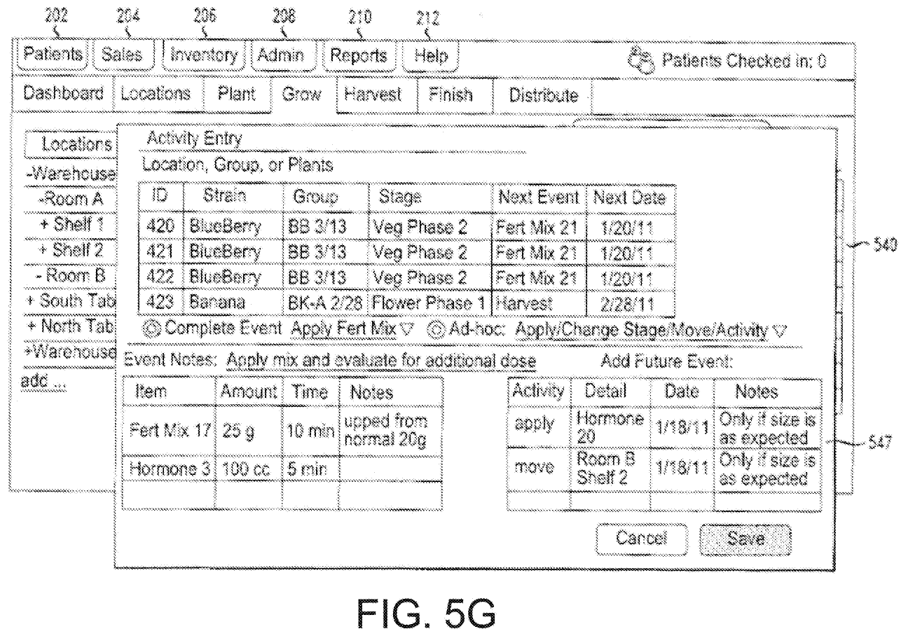

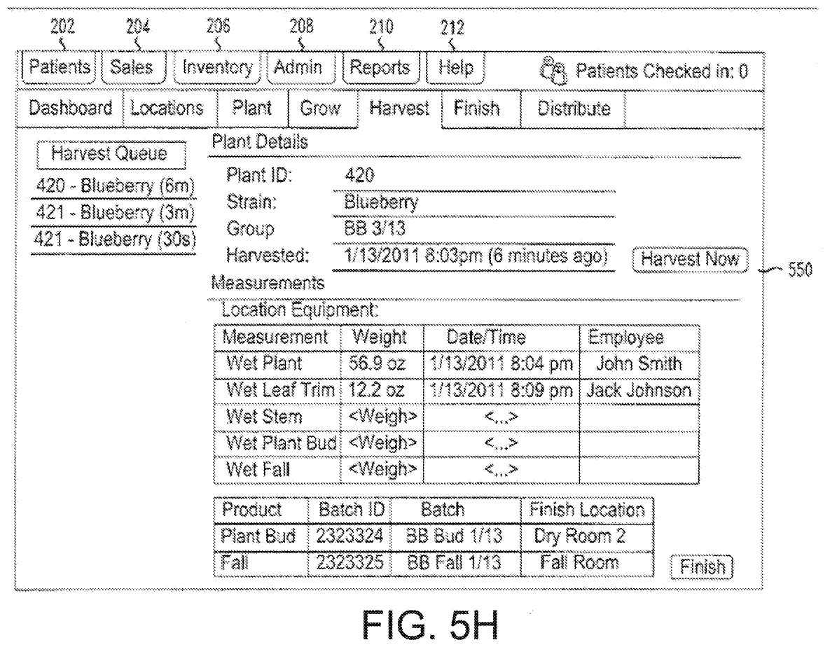

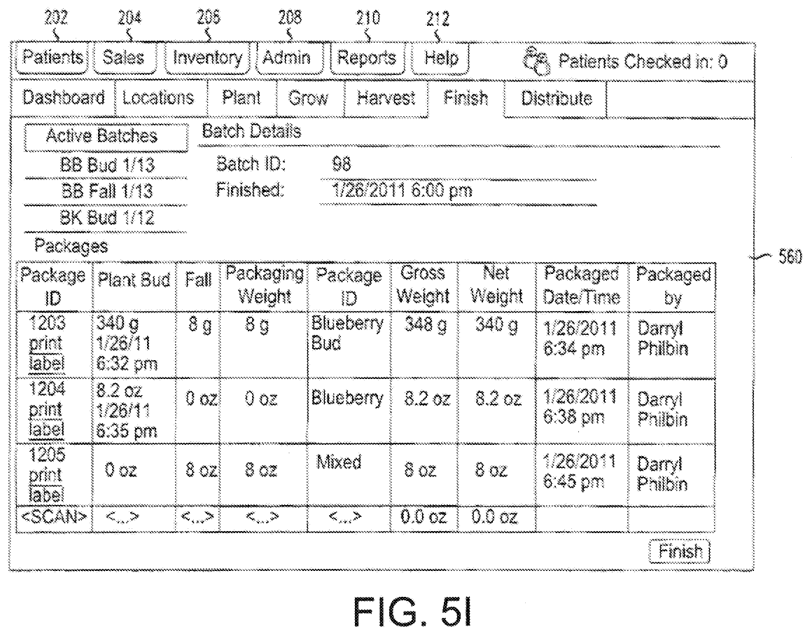

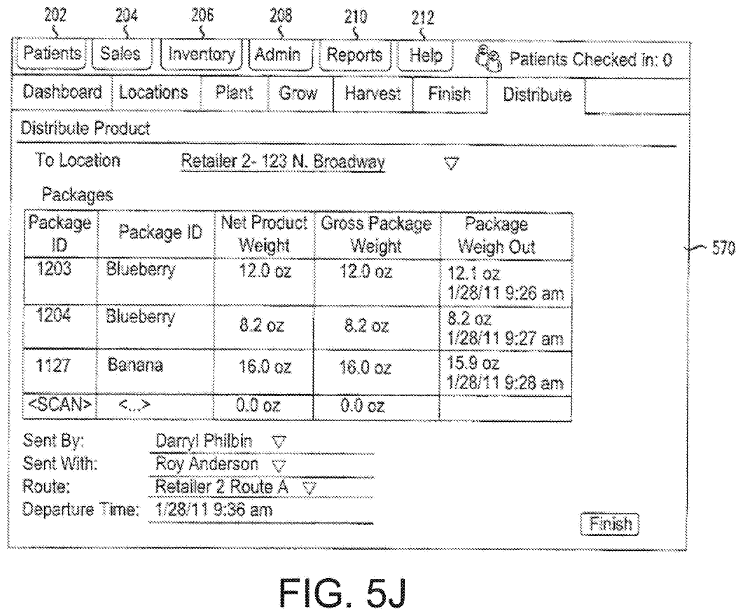

FIGS. 5A-5J illustrate exemplary screen displays 510-570 that may be used for tracking progression of a product through various stages from a raw form through processing and distribution. In the example shown in FIGS. 5A-5J, progression of a plant is tracked from planting through growth/processing and distribution according to an exemplary embodiment. These screens may be accessed by a user, e.g., at the growth-harvest-finish facility 120.

As with the screen displays shown in FIGS. 2-4T, access by a user to the screen displays 510-570 may be obtained by entering a username/password, and such access may be timed and tracked. Also, the screen displays shown in FIGS. 5A-5J include the various tabs 202, 204, 206, 208, 210, and 212 included in the screen displays shown in FIGS. 2-4T. Although not shown, the screen displays shown in FIGS. 5A-5J may include an additional tab by which growth inventory information may be accessed to track the plant from planting through distribution. It should be appreciated, however, that this information may also be accessed via the inventory tab 206. Using the screens shown in FIGS. 5A-5J, in conjunction with the screens shown in FIGS. 2-4T, the inventory of a plant product is tracked from planting to distribution and sale (or other disposal) by plant and patient.

According to an exemplary embodiment, the screens shown in FIGS. 5A-5J are accessed by a user, such as a plant tender. The plant tender may be at a growth/harvest facility 120, which may be at the same place as the POS 110 or at a different place. The screens may also be accessed by any other authorized user, e.g., an authorized user at end devices 130 and/or 140.

Referring to FIG. 5A, a user accesses a medicinal plant growth “dashboard” tab and is presented with a screen 510. The screen 510 indicates the number of patients, the number of allowed plants, and the total number of current plants, including the number of plants in vegetation and the number of plants flowering. The plant status is also summarized by plant type and location. The start date of each plant, e.g., the planting date, the duration of the plant in the growth-harvest-finish process, e.g., in days, the stage of the plant (e.g., seed/clone, vegetation, flowering, trimming, and curing), and the extent of completion of growth-harvest-finish process for the plant, e.g., as a percentage.

Referring to FIG. 5B, by accessing the locations, location data of medicinal plants may be displayed via a screen 520. Such data may include the location capacity for various plant stages, including the number of plants at various stages, the equipment at a location, and location notes. This information may be updated by the plant tender or another authorized user. While FIG. 5B illustrates a warehouse location, it should be appreciated that a plant location may be tracked in an outside location, e.g., a field.

Referring to FIG. 5C, detailed information regarding plants at a particular location is provided with reference to plant ids via a plant tab, causing the screen 530 to be displayed. Each plant id is associated with a specific patient. A label. e.g., a bar code label or an RFID tag, with the plant id may be attached to each plant and/or plant container for tracking the plant. According to an exemplary embodiment, the same plant id may be used from planting through disposal of the plant and any products resulting from processing of the plant.

The plants may be further organized by group and strain. The date of planting (including seeding/cloning) may be displayed as a “born on” date, along with the harvest date. This information may be updated and edited by the plant tender or another authorized user. For example, as shown in FIG. 5D, plants may be added via dialog box 535. Using the dialog box 535, the plant tender may add plants and information regarding the growth schedule of the plants. The plant id may be added, e.g., by scanning a bar code affixed to the plant or plant container via scanner at the growth-harvest-finish facility 120.

Referring to FIG. 5E, selecting a growth tab causes the screen display 540 shown in FIG. 5E to be presented. Using this screen, a plant tender may record a monitoring entry by selecting a plant and a record monitoring entry button 542 and may log activity by selecting a plant and a button 544. Selecting the monitoring entry button 542 causes the dialog box 545 shown in FIG. 5F to be displayed. Using the dialog box 545, the plant tender may monitor and/or update the growing conditions, including, e.g., the temperature, humidity, pH level, CO₂ level, water level. This information may be automatically populated, e.g., via sensors measuring the growing conditions at the growth location, and/or entered manually or adjusted by a user, e.g., a plant tender. The user id and a timestamp may be automatically populated.

Referring again to FIG. 5E, selecting the button 544 causes the dialog box 547 shown in FIG. 5G to be displayed. Using this dialog box, the plant tender may review and update the plant id, strain, group, stage and next event/next date and make event notes. The plant tender may also add future events, e.g., apply a hormone or move a plant.

Referring to FIG. 5H, selecting a harvest tab causes a screen 550 to be displayed, showing plant details at harvest, including the plant id, the strain, the group, and the date and time harvested. The employee harvesting the plant may also be identified. Measurements of the plant and/or portions of the plant, e.g., the leaf, stem and portions of the plant's product that may fall off the plant, may be taken, e.g., by weighing the plant via a scale and the growth-harvest-finish facility 120. The measurements may be indicated on the screen shown in FIG. 5H, in addition to information indicating a product resulting from processing the plant (e.g., drying and curing the plant). The product may be identified by type, batch id, and batch type. The finishing location may also be provided.

Referring to FIG. 5I, selecting the finish tab causes display of a screen 560 to be displayed showing finished batch details including the batch ID and the finish date. Details regarding finished packages from a batch are summarized by packet id. Information regarding weights of portions of the product, e.g., the bud of the plant and portions of the plant that may fall off, is provided, along with packing weight, and product id. The weight of the actual product, including all portions of the product, is provided as the net weight. The net weight plus the packaging weight is reported as the gross weight. The weight may be reported in grams, ounces, or any other increment of weight desired. The package date/time and the packager are also indicated. The package id may be printed by a printer at the growth-harvest-finish facility 120 and affixed to the package.

Referring to FIG. 5J, the plant tender may select the distribute tab to be presented with a screen 570 that may be used to review information regarding the place where the product is to be distributed, i.e., the POS 110. The distribute tab may be selected to fulfill orders by the POS 110 and/or in response to an indication that patients are checked in. The address of the POS 110 may be given, along with a summary of the packages sent, including the package weight out, date, and time. The person distributing the plant and the person carrying the plant may be provided, along with the route and the departure time and date. Thus, the packaged product may be tracked from the growth-harvest-finish facility to the POS 110. From the POS 110, the disposal of the product is tracked as described above.

According to exemplary embodiments, information provided via the screen displays 5A-5J may be tracked and maintained by the inventory/patient tracking system 160 at any time during the growth-harvest-finish process. In this manner, each medicinal plant is tracked in association with a specific patient as it progresses from planting through distribution to a POS 110 from the growth-harvest-finish facility 120. After distribution, the disposal of the plant from the POS 110, e.g., by sale, is then tracked by the inventory/patient tracking system 160.

At any time during the growth/harvest/finish process, the plant may be disposed of before it is distributed, e.g., by destruction, by theft, by death, etc. This disposal is also tracked by the inventory/patient tracking system 160.

Once the plant is disposed of, the plant ID may be assigned to a new plant. The patient associated with the plant ID may be associated with the new plant or with another plant. According to an exemplary embodiment, at all times, each plant is associated with a specific patient.

According to exemplary embodiments, precise tracking of medicinal plant inventory is enabled at every stage from planting through distribution and disposal of the plant in association with specific patients. The plant may be tracked at each stage in increments as small as, e.g., 1/100^{th} of a gram. This precise inventory tracking enables compliance with government regulations while maximizing benefits and profits to the medicinal plant vendor and retailer (including profit and non-profit retailers).

Although not illustrated, it should be appreciated that selection of the help tab 212 may cause appropriate screen displays to be presented to a user. For example, the help tab 212 may be used to access a screen for obtaining helpful tips and contact information for a customer service agent associated with the inventory/patient tracking system 160.

FIG. 6 is a block diagram of a device 600 with which a system for tracking plant inventory may be implemented according to an exemplary embodiment. The device 600 may be used to implement the inventory/patient tracking server 160 and/or the POS 110. The device 600 includes a processor 610 that receives information, such as sales data, patient information, inventory information, etc., and requests for such information. In the case of the inventory/patient tracking server 160, such information is received from, e.g., the POS 110, the growth-harvest-finish facility 120, and the end user devices 130 and 140 via I/O Data Ports 620. In the case of the POS 110, such information is received from, e.g., the scanner 108, the growth-harvest-finish facility 120, the end user devices 130 and 140, and the inventory/patient tracking server 160. The I/O Data Ports 620 can be implemented with, e.g., an interface including an antenna or other suitable type of transceiver through which data and signals may be transmitted and received over, e.g., a wired connection, wireless connection, and/or the network 150. It should be appreciated that the I/O Data Ports 620 can be used for communications between, e.g., the POS 110, the growth-harvest-finish facility 120, and the end user devices 130 and 140 and the inventory/patient tracking server 160 via the network 150.

The processor 610 communicates with a memory 630 via, e.g., an address/data bus (not shown). The processor 610 can be any commercially available or customer processor. The memory 630 is representative of the overall hierarchy of memory devices containing the software and data used to implement the functionality of the device 600. The memory 630 can include, but is not limited to, the following types of devices: processor registers, processor cache, RAM, ROM, PROM, EPROM, EEPROM, flash memory, SRAMD, DRAM, other volatile memory forms, and non-volatile, semi-permanent or permanent memory types; for example, tape-based media, optical media, solid state media, hard disks, combinations thereof, and the like.

As shown in FIG. 6, the memory 630 may include several categories of software and data used in the device 600, including applications 640, a database 650, an operating system (OS) 660, and the input/output (I/O) device drivers 670. As will be appreciated by those skilled in the art, the OS 660 may be any operating system for use with a data processing system. The I/O device drivers 670 may include various routines accessed through the OS 660 by the applications 640 to communicate with devices, and certain memory components. The applications 640 can be stored in the memory 630 and/or in a firmware (not shown) as executable instructions, and can be executed by the processor 610. The applications 640 include various programs that, when executed by the processor 610, implement the various features of the device 600, including an application for tracking inventory and patient records, including maintaining and updating such inventory and patient records in compliance with government regulations, and one or more applications for encrypting/decrypting data. The applications 640 may be applied to data stored in the database 650, along with data received, e.g., via the I/O data ports 620. The database 650 represents the static and dynamic data used by the applications 640, the OS 660, the U/O device drivers 670 and other software programs that may reside in the memory. The database 650 may include, for example, inventory information, government regulations information, patient data, and encryption key(s) and ciphers.

While the memory 630 is illustrated as residing proximate the processor 610, it should be understood that at least a portion of the memory 630 can be a remotely accessed storage system, for example, a server on a communication network, a remote hard disk drive, a removable storage medium, combinations thereof, and the like. Thus, any of the data, applications, and/or software described above can be stored within the memory 630 and/or accessed via network connections to other data processing systems (not shown) that may include a local area network (LAN), a metropolitan area network (MAN), or a wide area network (WAN), for example.

It should be understood that FIG. 6 and the description above are intended to provide a brief, general description of a suitable environment in which the various aspects of some embodiments of the present disclosure can be implemented. While the description refers to computer-readable instructions, embodiments of the present disclosure also can be implemented in combination with other program modules and/or as a combination of hardware and software in addition to, or instead of, computer readable instructions. The term “application,” or variants thereof, is used expansively herein to include routines, program modules, programs, components, data structures, algorithms, and the like. Applications can be implemented on various system configurations, including single-processor or multiprocessor systems, minicomputers, mainframe computers, personal computers, hand-held computing devices, microprocessor-based, programmable consumer electronics, combinations thereof, and the like.

FIG. 7 illustrates a method for tracking plant inventory according to an exemplary embodiment. According to an exemplary embodiment, various steps of this method may be performed by the inventory/patient tracking system 160 and/or the POS 110. It should be understood that the steps or other interactions of the illustrated method are not necessarily presented in any particular order and that performance of some or all the steps in an alternative order is possible and is contemplated. The steps have been presented in the demonstrated order for ease of description and illustration. Steps can be added, omitted, and/or performed simultaneously without departing from the scope of the appended claims. It should also be understood that the method can be ended at any time. In certain embodiments, some or all steps of the method, and/or substantially equivalent steps can be performed by execution of computer-executable instructions stored or included on a non-transitory computer-readable medium.

Referring to FIG. 7, a specific plant is associated with a specific consumer, e.g., a specific patient or authorized representative of the patient, at step 710. Information identifying the specific consumer may be obtained by and received from a scanner as described above. At step 715, the specific consumer data is safeguarded, e.g., by encrypting the specific consumer data for storage in the database 650. Similarly, plant inventory data, item inventory data and other data may be encrypted for storage. At step 720, progression of plant through multiple stages including growth of the plant from planting, to processing of the plant into a product, e.g., at the growth-harvest-finish facility 120, and to distribution of the plant or the product resulting from processing the plant is monitored in association with a specific consumer. Progression of the plant from planting (e.g., seeding or cloning) may include various growth stages, such as vegetation, flowering, trimming, and curing. Processing of the plant into a product may include harvesting, finishing, and packaging. Distribution of the plant may include delivery from the growth-harvest-finish facility 120 to a POS 110. At step 730, disposal of the plant, e.g., by sale at the POS 110, is tracked. Disposal may include not only sale of a product resulting from processing of the plant but also death of the plant, destruction of the plant, theft of the plant, etc. Thus, it should be appreciated that monitoring of the plant progression in step 720 may end at any stage during the growth process before product processing or before distribution, e.g., if disposal of the plant occurs before product processing or distribution occurs. At step 740, the plant inventory is updated based upon the disposal of the plant. At step 750, the specific consumer data is encrypted for transmission as described above. Updated plant inventory data, item inventory data, and other data may be encrypted for transmission in a similar manner. At step 760, the encrypted specific consumer data is transmitted, along with the updated plant inventory to a central server via, e.g., the Internet, for maintenance in accordance with government regulations.

The law does not require, and it is economically prohibitive to illustrate and teach every possible embodiment of the present claims. Hence, the above-described embodiments are merely exemplary illustrations of implementations set forth for a clear understanding of the principles of the invention. Variations, modifications, and combinations may be made to the above-described embodiments without departing from the scope of the claims. All such variations, modifications, and combinations are included herein by the scope of this disclosure and the following claims.

What is claimed is:

1. A method, comprising:
obtaining, by a scanner associated with a retail store, information identifying a specific patient and disposal information associated with a specific medicinal plant, the specific medicinal plant identified by a unique plant id;
receiving, by a processor, information identifying the specific patient from the scanner and the disposal information associated with the specific medicinal plant;
encrypting, by the processor, the information identifying the specific patient for storage in accordance with the Health Insurance Portability and Accountability Act (HIPAA);
weighing, by at least one National Type Evaluation Program (NTEP) certified scale, the specific medicinal plant at multiple stages of progression of the specific medicinal plant from a raw form, the multiple stages including planting, growth, application of an additive, harvesting, finishing, assembling into at least one product, packaging, and delivery;
monitoring, by the processor, in association with the specific patient, progression of the specific medicinal plant, wherein monitoring includes receiving, from the at least one NTEP certified scale, a weight of the specific medicinal plant at each of the multiple stages;
tracking, by the processor, in association with the specific patient, disposal of at least a portion of the raw form of the specific medicinal plant and at least a portion of the product resulting from assembling the specific medicinal plant, wherein tracking includes receiving disposal information from the scanner associated with at least a portion of the raw form of the specific medicinal plant and at least a portion of the product resulting from assembling the specific medicinal plant;
updating, by the processor, inventory of the specific medicinal plant based on the disposal of the portion of the raw form of the specific medicinal plant and the portion of the product resulting from assembling the specific medicinal plant; and
transmitting, by a transceiver, the encrypted information identifying the specific patient and updated inventory information to a central server via the Internet for maintenance in accordance with government regulations,
the method to track inventory of the specific medicinal plant in association with the specific patient at each of the multiple stages through disposal with 1/100^{th} gram precision.

2. The method of claim 1, further comprising:
encrypting the information identifying the specific patient for transmission.

3. The method of claim 1, further comprising tracking the patient data.

4. The method of claim 1, wherein disposal of the portion of the specific medicinal plant includes sale of the portion of the product to the specific patient, and wherein updating inventory of the specific medicinal plant includes deducting an amount of the product upon sale of the portion of the product.

5. The method of claim 1, wherein the disposal of multiple portions of the raw form and multiple portions of products resulting from assembling the specific medicinal plant are tracked.

6. The method of claim 4, wherein the amount of the product deducted from inventory is greater or less than an amount of the product for which the specific patient is charged.

7. The method of claim 4, wherein a price of the portion of the product is based on at least one of a weight of the portion of the product and the specific patient to which the portion of the product is sold.

8. The method of claim 1, further comprising printing a label containing the unique plant id, wherein the label is attached to the specific medicinal plant or to a container for the specific medicinal plant for monitoring progression of the specific medicinal plant through the multiple stages and tracking the disposal of the portion of the raw form of the specific medicinal plant and the portion of the product resulting from assembling the specific medicinal plant.

9. The method of claim 1, further comprising, upon disposal, printing a label containing summary information regarding the specific medicinal plant, wherein the label is attached to a container of the portion of the raw form of the specific medicinal plant or the portion of the product resulting from assembling the specific medicinal plant.

10. A system, comprising:
a scanner associated with a retail store, the scanner to obtain information identifying a specific patient and disposal information associated with a specific medicinal plant, the specific medicinal plant identified by a unique plant id;
at least one National Type Evaluation Program (NTEP) certified scale to weigh the specific medicinal plant at multiple stages of progression of the specific medicinal plant from a raw form, the multiple stages including planting, growth, application of an additive, harvesting, finishing, assembling into at least one product, packaging, and delivery;
a processor,
a memory having stored thereon instructions which, when executed by the processor, cause the processor to perform:
receiving the information from the scanner identifying the specific patient and the disposal information associated with the specific medicinal plant;
encrypting the information identifying the specific patient for storage in accordance with the Health Insurance Portability and Accountability Act (HIPAA);
monitoring, in association with the specific patient, progression of the specific medicinal plant, wherein monitoring includes receiving, from the at least one NTEP certified scale, a weight of the specific medicinal plant at each of the multiple stages;
tracking, in association with the specific patient, disposal of at least a portion of the raw form of the specific medicinal plant and at least a portion of the product resulting from assembling the specific medicinal plant, wherein tracking includes receiving, from the scanner, disposal information associated with at least a portion of the raw form of the specific medicinal plant and at least a portion of the product resulting from assembling the specific medicinal plant;
updating inventory of the specific medicinal plant based on the disposal of the portion of the raw form of the specific medicinal plant and the portion of the product resulting from assembling the specific medicinal plant; and
a transceiver to transmit the encrypted information identifying the specific patient and updated inventory information to a central server via the Internet for maintenance in accordance with government regulations,
the system to track inventory of the specific medicinal plant in association with the specific patient at each of the multiple stages through disposal with 1/100^{th} gram precision.

11. The system of claim 10, wherein the instructions further cause the processor to encrypt the information identifying the specific patient for transmission.

12. The system of claim 10, wherein the instructions further cause the processor to track the patient data.

13. The system of claim 10, wherein disposal of the portion of the product includes sale of the portion of the product to the specific patient, and wherein updating inventory of the specific plant includes deducting an amount of the product from inventory upon sale of the portion of the product.

14. The system of claim 13, wherein the amount of the product deducted from inventory is greater or less than an amount of the product for which the specific patient is charged.

15. The system of claim 13, wherein a price of the portion of the product is based on at least one of a weight of the portion of the product and the specific patient to which the portion of the product is sold.

16. A non-transitory storage medium upon which computer-executable instructions are stored which, when executed by a processor, cause the processor to perform operations comprising:
receiving, from a scanner associated with a retail store, information identifying a specific patient and disposal information associated with a specific medicinal plant;
encrypting the information identifying the specific patient for storage in accordance with the Health Insurance Portability and Accountability Act (HIPAA);
monitoring, in association with the specific patient, progression of the specific medicinal plant, identified by a unique plant id, through multiple stages from a raw form, wherein the multiple stages include planting, growth, application of an additive, harvesting, finishing, assembling into at least one product, packaging and delivery, wherein monitoring includes receiving, from at least one National Type Evaluation Program (NTEP) certified scale that weighs the specific medicinal plant at each of the multiple stages, a weight of the specific medicinal plant at each of the multiple stages;
tracking, in association with the specific patient, disposal of at least—a portion of the raw form of the specific medicinal plant and at least a portion of the—product resulting from assembling the specific medicinal plant, wherein tracking includes receiving, from the scanner, disposal information associated with at least a portion of the raw form of the specific medicinal plant and at least a portion of the product resulting from assembling the specific medicinal plant;
updating inventory of the specific medicinal plant based on the disposal of the portion of the raw form of the specific medicinal plant and the portion of the product resulting from assembling the specific product; and
presenting the encrypted information identifying the specific patient and updated inventory information for transmission by a transceiver to a central server via the Internet for maintenance in accordance with government regulations,
the operations to track inventory of the specific medicinal plant in association with the specific patient at each of the multiple stages through disposal with 1/100th gram precision.

17. The non-transitory storage medium of claim 16, wherein the instructions, when executed by the processor, further cause the processor to encrypt the information identifying the specific patient for transmission.

18. The non-transitory storage medium of claim 16, wherein the instructions, when executed by the processor, further cause the processor to perform tracking the patient data.

20. The non-transitory storage medium of claim 16, wherein the disposal of multiple portions of the raw form and multiple portions of products resulting from assembling the specific medicinal plant are tracked.

19. The non-transitory storage medium of claim 16, wherein disposal of the portion of the product includes sale of the portion of the product to the specific patient, and updating inventory of the specific medicinal plant includes deducting an amount of the product from inventory upon sale of the portion of the product.

20. The non-transitory storage medium of claim 19, wherein the amount of the product deducted from inventory is greater or less than an amount of the product for which the specific patient is charged.
